SEQUENCE LISTING (50 sequences)

SEQ ID NO: 1 - DNA, 75 bp, Artificial
  Primer
accacagtacacacccgtttcccgccggagccgaatggctatctgcatattggccatgcgaaatctatctgcctg

SEQ ID NO: 2 - DNA, 75 bp, Artificial
  Primer
aacttcgggatcgcccaggactataaaggccagtgcaacctgcgtttcgacgacactaacccggtaaaagaagat

SEQ ID NO: 3 - DNA, 75 bp, Artificial
  Primer
atcgagtatgttgagtcgatcaaaaacgacgtagagtggttaggttttcactggtctggtaacgtccgttactcc

SEQ ID NO: 4 - DNA, 75 bp, Artificial
  Primer
atgcgcgatccggtgctgtaccgtattaaatttgctgaacaccaccagactggcaacaagtggtgcatctacccg

SEQ ID NO: 5 - DNA, 75 bp, Artificial
  Primer
atgtacgacttcacccactgcatcagcgatgccctggaaggtattacgcactctctgtgtacgcttgagttccag

SEQ ID NO: 6 - DNA, 75 bp, Artificial
  Primer
gacaaccgtcgtctgtacgactgggtactggacaacatcacgattcctgttcacccgcgccagtatgagttctcg

SEQ ID NO: 7 - DNA, 22 bp, Artificial
  Primer
accacagtacacacccgtttcc

SEQ ID NO: 8 - DNA, 23 bp, Artificial
  Primer
caggcagatagatttcgcatggc

SEQ ID NO: 9 - DNA, 20 bp, Artificial
  Primer
aacttcgggatcgcccagga

SEQ ID NO: 10 - DNA, 26 bp, Artificial
  Primer
atcttcttttaccgggttagtgtcgt

SEQ ID NO: 11 - DNA, 28 bp, Artificial
  Primer
atcgagtatgttgagtcgatcaaaaacg

SEQ ID NO: 12 - DNA, 22 bp, Artificial
  Primer
ggagtaacggacgttaccagac

SEQ ID NO: 13 - DNA, 18 bp, Artificial
  Primer
atgcgcgatccggtgctg

SEQ ID NO: 14 - DNA, 22 bp, Artificial
  Primer
cgggtagatgcaccacttgttg

SEQ ID NO: 15 - DNA, 24 bp, Artificial
  Primer
atgtacgacttcacccactgcatc

SEQ ID NO: 16 - DNA, 23 bp, Artificial
  Primer
ctggaactcaagcgtacacagag

SEQ ID NO: 17 - DNA, 21 bp, Artificial
  Primer
gacaaccgtcgtctgtacgac

SEQ ID NO: 18 - DNA, 20 bp, Artificial
  Primer
cgagaactcatactggcgcg

SEQ ID NO: 19 - DNA, 96 bp, Artificial
  Primer
gatcccgcgaaattaatacgactcactatagggagaccacaacggtttccctctagaaataattttgtttaactttaagaaggagatgccaccatg

SEQ ID NO: 20 - DNA, 93 bp, Artificial
  Primer
gcttccggcggggccgctgcgctgtctggtgccctgtccatcagcgctgtcggttctctgtccttgatcggcgtgatcctcggcgctggagga

SEQ ID NO: 21 - DNA, 24 bp, Artificial
  Primer
gatcccgcgaaattaatacgactc

SEQ ID NO: 22 - DNA, 24 bp, Artificial
  Primer
catggtggcatctccttcttaaag

SEQ ID NO: 23 - DNA, 18 bp, Artificial
  Primer
ggtggtgctgctgcgctg

SEQ ID NO: 24 - DNA, 21 bp, Artificial
  Primer
gcctccagcgccgaggatcac

SEQ ID NO: 25 - DNA, 26 bp, Artificial
  Primer
atgatcaaacgctctaagaagaacag

SEQ ID NO: 26 - DNA, 21 bp, Artificial
  Primer
ctcggaatagagtatgggggg

SEQ ID NO: 27 - DNA, 23 bp, Artificial
  Primer
tatgatcctaccagacccttcag

SEQ ID NO: 28 - DNA, 17 bp, Artificial
  Primer
tggcaccctcttcgccc

SEQ ID NO: 29 - DNA, 22 bp, Artificial
  Primer
ggctttgtggatttgaccctcc

SEQ ID NO: 30 - DNA, 21 bp, Artificial
  Primer
gcgccagacgagaccaatcat

SEQ ID NO: 31 - DNA, 21 bp, Artificial
  Primer
tccatggagcacccagtgaag

SEQ ID NO: 32 - DNA, 21 bp, Artificial
  Primer
gtccaagagcaagttaggagc

SEQ ID NO: 33 - DNA, 24 bp, Artificial
  Primer
aggaaccagggaaaatgtgtagag

SEQ ID NO: 34 - DNA, 22 bp, Artificial
  Primer
catgcggaaccgagatgatgta

SEQ ID NO: 35 - DNA, 24 bp, Artificial
  Primer
atgaatctgcagggagaggagttt

SEQ ID NO: 36 - DNA, 31 bp, Artificial
  Primer
agaattaagcaaaataatagatttgaggcac

SEQ ID NO: 37 - DNA, 23 bp, Artificial
  Primer
ggagtgtacacatttctgtccag

SEQ ID NO: 38 - DNA, 21 bp, Artificial
  Primer
tgccttggccatcaggtggat

SEQ ID NO: 39 - DNA, 16 bp, Artificial
  Primer
ggcctgaccctgcagc

SEQ ID NO: 40 - DNA, 21 bp, Artificial
  Primer
catgtgcctgatgtgggagag

SEQ ID NO: 41 - DNA, 24 bp, Artificial
  Primer
agtaacaaaggcatggagcatctg

SEQ ID NO: 42 - DNA, 22 bp, Artificial
  Primer
caccacgttcttgcacttcatg

SEQ ID NO: 43 - DNA, 20 bp, Artificial
  Primer
cccctctatgacctgctgct

SEQ ID NO: 44 - DNA, 19 bp, Artificial
  Primer
gctagtgggcgcatgtagg

SEQ ID NO: 45 - DNA, 84 bp, Artificial
  Primer
atctcgatcccgcgataatacgactcactatagggacaattactatttacaattacaatggactacaaagatgacgacgataag

SEQ ID NO: 46 - DNA, 96 bp, Artificial
  Primer
aagcgacgatggaaaaagaatttcatagccgtctcagcagccaaccgctttaagaaaatctcatcctccggggcacttcatcaccatcaccatcac

SEQ ID NO: 47 - DNA, 24 bp, Artificial
  Primer
atctcgatcccgcgataatacgac

SEQ ID NO: 48 - DNA, 30 bp, Artificial
  Primer
cttatcgtcgtcatctttgtagtccattgt

SEQ ID NO: 49 - DNA, 28 bp, Artificial
  Primer
aagcgacgatggaaaaagaatttcatag

SEQ ID NO: 50 - DNA, 40 bp, Artificial
  Primer
gtgatggtgatggtgatgaagtgccccggaggatgagatt

(19) United States
(12) Patent Application Publication
(10) Pub. No.: US 2004/0091920 A1
(43) Pub. Date: May 13, 2004

(54) Method of constructing a mutant DNA library and use thereof

(75) Inventors: Toru Tsuji, Kanagawa (JP); Hiroshi Yanagawa, Kanagawa (JP)

Correspondence Address: GREENBLUM & BERNSTEIN, P.L.C., 1950 ROLAND CLARKE PLACE, RESTON, VA, 20191, US

(73) Assignee: Mitsubishi Chemical Corporation, Tokyo (JP)

(21) Appl. No.: 10/637,009

(22) Filed: Aug. 8, 2003

Related U.S. Application Data
(63) Continuation-in-part of application No. 10/396,334, filed on Mar. 26, 2003.
Application No. 10/396,334 is a continuation of application No. PCT/JP01/08387, filed on Sep. 26, 2001.

(30) Foreign Application Priority Data
Sep. 27, 2000 (JP) 2000-293692
Feb. 6, 2001 (JP) 2001-29138

(51) Int. Cl. C12Q 1/68; C12P 19/34

(52) U.S. Cl. 435/6; 435/91.2

(57) ABSTRACT

The present invention provides a method of constructing a DNA library comprising: (a) ligating building block DNAs in any combination; (b) selecting and mixing the obtained ligated building block DNAs such that all ligated building block DNAs to be used have an end sequence that overlaps with an end sequence of at least one other type of ligated building block DNA; and (c) performing Polymerase Chain Reaction using the mixture of ligated building block DNAs as a template to obtain a DNA library comprising 2 or more clones. The present invention provides a method of constructing a group of genes formed by ligation of a plurality of DNA fragments encoding any amino acid sequence in various orders and lengths.

### CROSS-REFERENCE TO RELATED APPLICATION

This application is a continuation-in-part of Application No. 10/396,334, filed Mar. 26, 2003, which is a continuation of International Application No. PCT/JP01/08387, filed Sep. 26, 2001, which was not published in English, which claims priority under 35 U.S.C. § 119 of Japanese Application Nos. 2000-293692 filed Sep. 27, 2000 and 2001-029138 filed Feb. 6, 2001, the disclosures of which are expressly incorporated by reference herein in their entireties.

### TECHNICAL FIELD

The present invention relates to a method of constructing a DNA library, which comprises performing a Polymerase Chain Reaction (hereinafter, also referred to as “PCR”) using a mixture of ligated building block DNAs as a template. More specifically, the present invention relates to a method of constructing a mutant DNA library which comprises performing PCR using, as a template, a mixture of ligated building block DNAs formed by ligating DNA sequences encoding specific amino acid sequences such as modules, secondary structures, super-secondary structure or motifs of protein, DNA constituting primitive sequences, exons, or expression control regions, or DNA encoding artificial amino acid sequences, etc.; and to a protein library obtained by transcription and translation of the DNA library. Further, the present invention relates to a method of obtaining a mutant protein having a desired property which comprises selecting a protein having a desired property using the protein library, and to a protein having a further enhanced desired property which is obtained by application of evolutionary molecular engineering techniques.

### BACKGROUND ART

At present, many enzymes are used in industrial bioprocess. From their relevance to environmental issues, the further future diffusion of bioprocesses having high energy efficiency is anticipated. Further, accompanying the development of genetic engineering, it is thought that demand for proteins as medicaments and biosensors will increase. However, many proteins have low structural stability and are easily denatured by heat, organic solvents or th like. Further, aggregation in the manufacturing process is a cause of lowered rates of recovery of synthesis products. Such denaturation and aggregation are often irreversible, and once a protein is denatured, it is difficult to restore the protein to its natural functional state.

For this reason, attempts are being made to improve thermal resistance and organic solvent resistance, etc by protein engineering or evolutionary molecular engineering methods. For example, in protein engineering, methods are being used which involve predicting sites that appear likely to impart stability to the structure of proteins with known structures, and performing amino acid substitution (Oyokagukukoza: “Tanpakushitu Kogaku”, Asakurashoten, Katuhide Iketani, Haruki Nakamura (Ed.), 1991). Further, by using evolutionary molecular engineering techniques, amino acid substitution is performed site specifically or randomly, and by selection using an increase in the target property as an indicator, a protein having improved function can be obtained.

Evolutionary molecular engineering is a superior method of creating a protein having a specific function. In evolutionary molecular engineering of proteins, first, a group of mutants comprising many proteins (hereinafter, also referred to as “mutant protein library”) is constructed. Next, a mutant protein having the target function is selected from the library. Further, mutations are introduced into DNA encoding the selected mutant protein, this mutant is expressed and a mutant protein library is constructed again, and a mutant protein having a further enhanced desired function, is selected. By repeating this operation several times, a protein having a superior function can be obtained.

To obtain a mutant protein according to such an evolutionary molecular engineering, it is essential that mutant protein library and a mutant DNA library encoding these proteins, can screen a larger sequence space. Thus, techniques of constructing these libraries are extremely important elementary techniques for evolutionary molecular engineering.

Techniques for constructing a mutant DNA library were in the initial period limited to using mutagenic agents (Myers, R. M. et al. (1985) Science 229, 242-247; Chu, C.-T. et al. (1979) Virology 98, 168-181) or radiation (Billi, D. (2000) Appl. Environ. Microbiol. 66, 1489-1492) on DNA. However, accompanying the development of new techniques such as chemical synthesis of DNA (Sinha, N. D. et al. (1984) Nucleic Acids Res. 12, 4539-4557) and Polymerase Chain Reaction (Saiki, R. K. et al. (1985) Science 230, 1350-1354), methods of introducing a mutation, such as methods using recombinant techniques (Botstein, D. & Shortle, D. (1985) Science 229, 1193-1201), Error-prone PCR (Leung, D. W. et al, (1989) Technique 1, 11-15), and DNA shuffling (Stemmer, W. P. C. (1994) Proc. Natl. Acad. Sci. USA. 91, 10747-10751), etc., have become more dynamic both qualitatively and quantitatively.

In Error-prone PCR, by adding an excess amount of manganese ions and 4 types deoxyribonucleotide triphosphate at various mixing ratios, and performing PCR using Taq DNA polymerase, point mutations are introduced during replication of DNA.

In DNA shuffling, after introduction of point mutations by Error-prone PCR into a DNA encoding a protein to be subjected to evolution, the DNA is cleaved with DNase I. Thereafter, by performing PCR in the absence of a primer, recombination occurs between the homologous sequences, various point mutations that have already been introduced are mixed, thereby constructing a DNA library having various mutations introduced thereinto. That is, point mutations can be introduced more efficiently by DNA shuffling than by Error-prone PCR. Further, sequences within a family of proteins having relatively high sequence homology can be used as parent sequences for DNA shuffling.

A mutant DNA library where point mutations have been introduced to DNA can be prepared according to the above-described method. However, the sequence space of amino acids of the protein is extremely wide. For example, for a protein sequence consisting of 100 amino acid residues, there are 20¹⁰⁰≈10¹³⁰ possible sequences. This is more than the number of atoms in the universe (10⁷⁵). It is impossible that this enormous number of sequences has been exhaustively tested in the process of evolution in less than 4,000,000,000 years. In other words, there is a strong possibility that within this enormous space of amino acids sequences of proteins, there are sequences having useful functions which have not yet been tested by organisms present on the earth.

In this respect, with Error-prone PCR, only a very small number of point mutation can be introduced. Further, because with DNA shuffling it is only possible to introduce point mutations of sequences sharing high sequence homology, it is not possible to screen a broad sequence space of proteins by using a library constructed by this method.

Further, the method which can combine DNA sequence encoding amino acid sequence without the homologous region, not for introducing point mutations to DNA sequences, has not been known.

For example, a method of simply ligating arbitrary DNA fragments using DNA ligase could be considered as a method of recombining particular regions of DNA as mentioned above. However, with a ligation such as this, because DNA fragments ligated in the opposite direction would appear at a rate of 1 in 2, it would not be possible to obtain the amino acid sequence that the DNA fragments originally encoded, and further, because termination codons would frequently appear, it would be difficult to construct a structural gene corresponding to the smallest globular protein (consisting of approximately 100 amino acid residues). Thus, such a method would be unsuitable as a method of constructing a library.

Further, as a ligation method which preserves the directionality of DNA, a method involving digesting the 5′-end and 3′-end of the building block DNA to be ligated with different restriction enzymes and ligating these fragments with DNA ligase, could be considered. However, in this case, although the directionality of each building block is maintained, a recognition site (linker) of a restriction enzyme needs to be artificially added to each building block, and as a result, there arises the problem that specific amino acids are inserted in the original amino acid sequence.

The present inventors have disclosed (JP Patent Publication (Unexamined Application) No. 10-014578) one method of introducing site specific mutations at the building block level. This method involves following steps: (1) The structural gene is divided into several building block DNAs, and isolated, (2) Builcing block DNA is isolated and ligated with arbitary combinations, (3) Ligated building block DNAs are selected and mixed to allow overlap extension at the end of building block DNAs to be rearranged into a building block sequence which contains the permutations of building block DNAs what we want to permuate, and (4) amplification is perfomed by using this ligated building block DNA as a template, and using the building block DNAs for both ends as primers. However, this method is directed to obtaining a permuted mutant DNA which is obtained by rearrangement of a predetermined number of building block DNAs, and further because only one type of mutant DNA can be obtained by a single PCR, it was not a method of constructing a library having a diverse protein sequence space by a single PCR.

### DISCLOSURE OF THE INVENTION

The object of the present invention is the efficient construction of a mutant DNA library by using, as a building block, a region constituting a part of a natural protein such as module, secondary structure, super-secondary structure or motif of protein, primitive sequence, or an exon of DNA, or region composed of an expression regulatory region or an artificial amino acid sequence (hereinafter, also called a “building block”), and ligating the DNA constituting these in an arbitrary combination, which is provided as a method of constructing a library for screening a large protein sequence space, which was not attained by means of Error-prone PCR or DNA shuffling which are conventional methods for constructing a mutant DNA library.

A further another object of the present invention is, as a method of obtaining a protein having a desired property using evolutionary molecular engineering techniques, to prepare a protein library by transcribing and translating the above described DNA library, and to select and obtain a protein having the desired property using the protein library.

Further, the present invention is directed to obtaining a mutant protein having a desired property by selecting the protein having the desired property from the protein library, and further to obtaining a protein wherein the desired property is further improved by evolutionary molecular engineering.

Further, the present invention is directed to obtaining DNA encoding a protein having a desired property by analyzing the DNA encoding the selected protein, and preparing a recombinant protein having the desired property from the DNA.

As a result of deliberate study directed to solving the above problem, the present inventors have discovered that by preparing and mixing a plurality of types of ligated building block DNA formed by ligating 2 DNA fragments encoding any amino acid sequence, and performing PCR using these as templates, then DNAs having various DNA sequences which are obtained by random recombination of DNA fragments encoding any amino acid sequence, would be synthesized in a single PCR (hereinafter referred to as “random library” at times). Further, the present inventors focused on the fact that in the protein synthesis system of eukaryotic organisms, a plurality of types of proteins are synthesized by combining the limited exons by alternative splicing. It was also found that, if ligated building block DNA was prepared by ligating building block DNAs obtained by segmenting a structural gene encoding a protein into arbitrary fragments, while preserving their order in the structural gene from upstream to downstream, and PCR is performed using these mixed at a specific ratio as template DNA, a group of DNA sequences arising from alternative splicing (hereinafter, this is also referred to as a “alternative splicing library”) could be constructed. The present invention was completed on the basis of thes findings.

Thus, according to the present invention, the following inventions are provided:

(1) A method of constructing a DNA library comprising:

(a) ligating building block DNAs in any combination;

(b) selecting and mixing the obtained ligated building block DNAs such that all ligated building block DNAs to be used have an end sequence that overlaps with an end sequence of at least one other type of ligated building block DNA; and

(c) performing Polymerase Chain Reaction using the mixture of ligated building block DNAs as a template to obtain a DNA library comprising 2 or more clones.

(2) The method according to (1) wherein, in step (b), the ligated building block DNAs are selected and mixed such that all ligated building block DNAs to be used have end sequences that overlap with the end sequence of at least one other type of ligated building block DNA, and such that either the 5′-end or 3′-end sequence of at least one type of ligated building block DNA overlaps with an end of at least two other types of ligated building block DNA.

(3) The method according to (1) or (2) wherein the mixture of ligated building block DNAs is a mixture of ligated building blocks formed by ligation of 2 building block DNAs in all combinations of the building block DNAs used,

(4) The method according to (1), wherein the building block DNAs are obtained from a structural gene encoding a protein which was segmented into L units (where L is an integer of 3 or more); wherein the method of ligation is such that the 3′-end of any building block DNA is ligated to the 5′-end of a building block DNA existing further toward the C-terminal side of the protein than the above building block DNA, in such a way that the amino acid sequence of each building block is preserved; and wherein the mixture of ligated building block DNAs is prepared by the classification according to X (where X is an integer between 0 and L-2, inclusive), which is the number of intervening building block DNAs between these block DNAs ligated in the structural gene, and by mixing with the proportions fulfilling the following formula (1):

_{L-X-}1C_{M-X} (1)

wherein, L and X have the same meaning as that described above, and M represents the difference between the total number of building blocks and the number of building blocks possessed by the mutant DNA to be prepared.

(5) The method according to (1), wherein the building block DNA results from segmentation of each DNA encoding a group of proteins having a similar 3-dimensional structure, but differing amino acid sequences, into L building blocks (where, L is an integer of 3 or more); wherein the method of ligation is such that the 3′-end of any building block DNA is ligated to the 5′-end of a building block DNA existing further toward the C-terminal side of the group of proteins than the above building block DNA in such a way that the amino acid sequence of each building block is preserved, and wherein the mixture of ligated building block DNAs is prepared by the classification according to X (where X is an integer between 0 and L-2, inclusive), which is the number of intervening building block DNAs between these block DNAs ligated in the structural gene, and by mixing with the proportions fulfilling the following formula (1):

_{L-X-1}C_{M-X} (1)

wherein the formula, L, X and M have the same meaning as that described above.

(6) The method according to any one of (1) to (5) wherein the Polymerase Chain Reaction is conducted in the presence of primers having sequences complementary to the building block DNA intended to form both ends of the DNA to be constructed.

(7) The method according to any one of (1) to (6) wherein the Polymerase Chain Reaction is performed in the presence of ligated building block DNA comprising a common DNA sequence, and a primer having a sequence complementary to the common DNA sequence.

(8) A mutant DNA library obtained by the method of any one of (1) to (7).

(9) A protein library obtained by incorporating the DNA library according to (8) into an expression vector, transforming a host cell with the expression vector, culturing the transformant, and collecting proteins from the culture product.

(10) An RNA library obtained by transcription of the DNA library according to (8).

(11) A method of preparing a protein library, which comprises expressing the DNA library according to (8) by use of the cell free transcription and/or translation system or the RNA library according to (10) by a cell free transcription system.

(12) A protein library obtained by the method of (11).

(13) The library of nucleic acid-protein ligated molecule whose proteins are encoded by DNA or RNA from the DNA library according to (7) or from RNA library according to (9).

(14) A method of obtaining a protein which comprises selecting a protein having a desired property using the library according to (9), (12) or (13),

(15) A protein obtained by the method according to (14).

(16) A method of obtaining a protein, which comprises.

(a) preparing DNA encoding a protein according to (15);

(b) introducing mutations into the DNA;

(c) amplifying the DNA into which mutations were introduced;

(d)preparing a protein library by transcription/translation of the amplified DNA; and

(e)obtaining a protein having a desired property using the library.

(17) A protein obtained by the method according to (16).

(18) A method of obtaining DNA encoding the protein according to (15) or (17).

(19) DNA obtained by the method according to (18).

### BRIEF EXPLANATION OF THE DRAWINGS

FIG. 1 indicates the concept of the present invention. A case where 3 types of building block DNA are shuffled is indicated as an example. By mixing ligated building block DNAs form d by ligation of 2 building block DNAs and performing PCR using T7 forward primer and Ex reverse primer, numerous DNA fragments in which the 3 types of building block DNA are ligated in various orders, are synthesized in a single PCR.

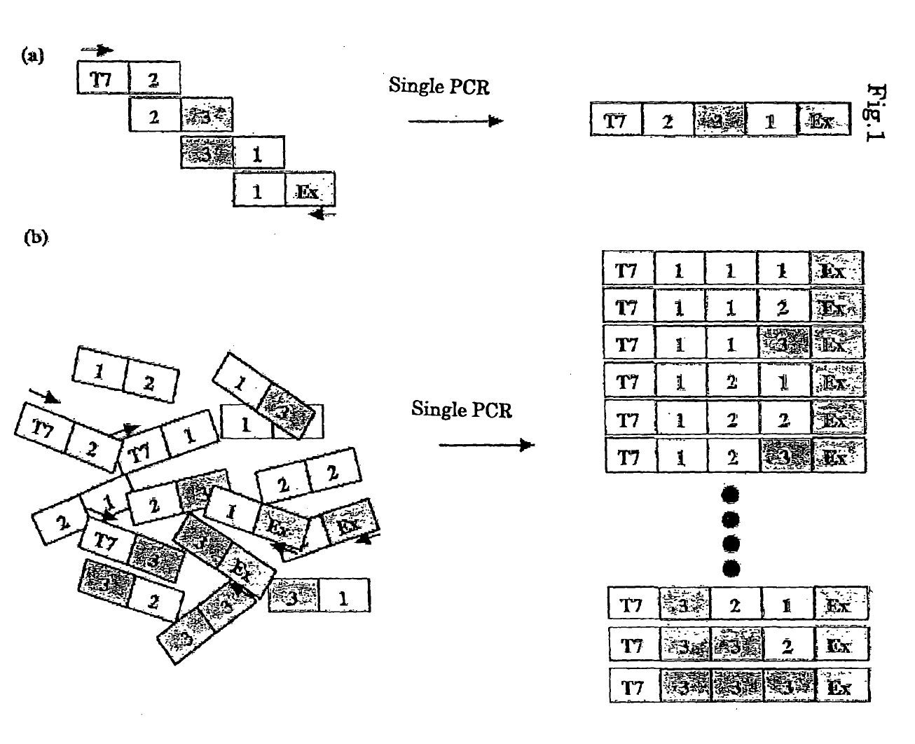

FIG. 2 is an electrophoretogram of the PCR products separated using an agarose gel (2%). Each lane contains 50 μl of PCR product. Sequences corresponding to the DNA bands of the ladder are shown on the right. M is a DNA marker.

FIG. 3 indicates the principle of the method of constructing a mutant DNA library according to the present invention.

FIG. 4 is a chart indicating a ligand binding domain of human estrogen receptor α, which was cleaved at the boundary of exons and secondary structures, and divided into 10 building blocks.

FIG. 5 indicates all ligated building block DNAs used for constructing the mutation gene library of the present invention from a structural gene consisting of 10 building blocks. Each ligated building block DNA can be classified into 9 ligated building block DNA groups from X=0 to X=8, as shown by the diagonal line (a). The frequency of each ligated building block DNA group in the group of mutant structural genes constituted by a certain number of building blocks, was showed in (b).

FIG. 6 is an agarose gel electrophoretogram of the separated PCR products for the construction of the mutant DNA library according to the present invention. FIG. 6(a) and (b) are PCR products where a group of structural genes constituted by 8 and 5 building block DNAs respectively, are constructed. In the figure, M indicates a marker.

FIG. 7 indicates the structure of DNAs given as block numbers in a mutant gene library of the present invention obtained by performing PCR under conditions whereby a structural gene is obtained from 8 building block DNAs.

FIG. 8 indicates the structure of DNAs given as block numbers in a mutant gene library of the present invention obtained by performing PCR under conditions whereby a structural gene is obtained from 5 building block DNAs.

### BEST MODE OF CARRYING OUT THE INVENTION

Below, the present invention is explained in further detail. It should be noted that except as where otherwise specifically indicated, the explanation is common to both random library and alternative splicing library.

(1) Building Block DNA

“Building block”, as used in the present invention, indicates a unit of specific region having some structural, functional, or evolutionary meaning in a protein, gene or genome DNA, or any region not having such a meaning or an expression regulatory region, etc. Regions having specific meaning in proteins specifically indicate, for example, protein modules, secondary structures, super secondary structures, structure motifs, motifs, primitive sequences, and artificial amino acid sequences, and in genes indicate exons. An expression regulatory region specifically refers to a core promoter element, upstream control element, enhancer promoter, or enhancer, etc.

Where a random library is to be prepared, any of the above described building blocks can be used, but in the case of an alternative splicing library, building blocks segmented from a structural gene encoding a protein are used.

A module of protein refers to a region which is continuous in primary structure and sterically compact and consists of 10 to 40 amino acid residues in a globular protein or domain. A polypeptide encoded by an exon of a eukaryotic organism corresponds well to a building block exhibiting a module structure, and a module is a basic polypeptide constituting a protein, thought to have been an important material in the early stages of the evolution of living organisms. (Go, M. et al. (1987) Cold Spring Harb. Symp. Quant. Biol., 52, 915-924). Specific examples include modules which constitute lysozyme, hemoglobin, triose phosphate isomerase, and bamase.

Further, secondary structure of a protein refers to, in the conformation structure of a protein, repeated regular structures such as α-helix, β-sheet, β-turn, or loop structure, which is special three dimensional structures observed in a relatively narrow scope (up to about 10 residues) formed by hydrogen bonds between C═O groups and NH groups in the main chain of the peptide, (Salemme, F. R. et al. (1983) In biochemistry, ed. Zubay, G., 69-129, Addison-Wesley Publishing Co., Massachusetts), chameleon sequence (Minor, D. L. et al., Nature, 380(6576), 730-734(1996)), and G-peptide (Honda, S. et al., J. Mol. Biol., 295(2), 269-278(2000)).

A super-secondary structure of a protein refers to a unit wherein several secondary structural units such as α-helix, β-sheet structures come together to form a compact three dimensional structure (Salemme, F. R. et al. (1983) In biochemistry, ed. Zubay, G, 69-129, Addison-Wesley Publishing co., Massachusetts). Specific examples include a helix loop-helix structure.

A motif of a protein refers to short but significantly conserved amino acid sequences when compared by an operation of arranging an amino acid sequence with a very similar amino acid sequence of a protein, or one that has similar properties, so that it is in the same position (sequence alignment). In other words, motif is present at specific sites of a protein, and it indicates amino acid sequences and nucleotide sequences that have been conserved in the process of evolution. Specific examples include Greek Key motif, etc., and motifs registered in the protein motif database, PROSITE (http://expasy.hcuge.ch/sprot/prosite.html), etc.

A primitive sequence refers to an amino acid sequence which corresponds to the arrangement of specificity of transfer RNA. It has been predicted from homology comprison of presently existing protein sequences that the primitive sequences played an important role for the diversification of protein function in the early stages of evolution, (Ohnishi, K. (1993) In Endocytobiology V, Eds.; S. Sato et al., Tubingen University Press, Tubingen, 407-414).

An exon refers to a nucleotide sequence within the gene DNA of a eukaryotic organism that is translated into the protein. Exons are segmented by intron nucleotide sequences that are not, translated into the protein. After genome DNA is transcribed into RNA, introns are excised by splicing, and exons only are joined together to become mature mRNA being the information for protein synthesis (Gilbert, W. (1978) Nature, 271, 501).

“Building block DNA” as used in the present invention refers to DNA constituting a building block. When the building block is produced by segmentation of a structural gene, and the nucleotide sequence possessed by the original structural gene is a singular one, then the building block DNAs obtained by segmentation thereof, each are ones having a singular nucleotide sequence. Further, building block is not obtained by segmentation of a structural gene, DNA included in a building block DNA means a DNA group comprising plurality of nucleotide sequences having homology. Here, a plurality of nucleotide sequences having homology refers to one wherein homology is sufficient to enable the PCR described hereinafter, and the reading frame of that DNA is retained.

Such a building block DNA consists of two complementary strands, and may be DNA cleaved and isolated from any DNA strand or may chemical synthesized DNA. Further, as long as the DNA strand is a unit having the above described structure there is no particular restriction on the DNA length, and fragments having a length of 30 to 120 bp encoding 10 to 40 amino acids, or a mixture of DNA fragments having a 5′- or 3′-end differing by ±3 to 15 bp, can be used. An example of a method of preparing such a DNA fragment having a 5′- or 3′-end differing by ±3 to 15 bp, is a method involving preparation by PCR using a plurality of primers respectively designed to comprise a sequence shifted ±3 to 15 bp on the adjacent nucleotide sequence.

In the method of constructing a mutant DNA library according to the present invention, firstly, this building block DNA is prepared. Preparation can be according to any method as long as the above described building block DNA can be obtained, but preferably building block DNA is obtained, for example, by PCR using DNA comprising the target DNA sequence as a template.

Further, the DNA can be obtained by annealing complementary strands of DNA prepared by chemical synthesis.

(2) Ligated Building Block DNA

The obtained building block DNAs are ligated using DNA ligase, etc. to prepare a building block DNA ligation product (hereinafter, this is also referred to as a “ligated building block DNA”). The ligated building block DNA, ligated in this manner by using DNA ligase, can be amplified by performing PCR using the ligated building block DNA as a template. At this time, it is necessary to amplify ligated building block DNAs in which each of the building block DNAs are ligated in such a direction that the reading frame of the amino acids is preserved. For this purpose, for example, where amplifying a ligated building block DNA denoted “1-2” formed by ligation of a “building block 1” and a “building block 2”, PCR is performed using a forward primer having complernentarity to the nucleotide sequence of the 5′-end of “building block 1” and a reverse primer having complementarity to the nucleotide sequence of the 3′-end of “building block 2”. By selecting primers in this manner, ligated building block DNA formed by inverted ligation by DNA ligase, etc, can be automatically removed.

After performing the reaction with DNA ligase, etc., or the above described PCR, the ligated building block DNA can be obtained by separating this reaction solution by agarose gel electrophoresis, etc., excising the portion of gel containing the target DNA, and collecting and purifying the DNA from the gel using known methods. Further, the full length DNA of the above described ligated building block DNA can be prepared and obtained by chemical synthesis.

Regarding methods of selection, and order of ligation of building block DNAs for preparing ligated building block DNA, in the case of preparation of a random library, any building block DNA can be used as building block DNA to be ligated, and there is no particular restriction on the number of units to be ligated. However, from the point of view of efficiency, etc. of variety of elements constituting the library to be constructed, it is preferable to prepare ligated building block DNA formed by ligation of 2 building block DNAs. There is no particular restriction on the building block DNA to be ligated, and the order of ligation thereof. However, if ligated building block DNAs are prepared with all combinations of building block DNAs used, then in the DNA library prepared by the method described hereinafter using this, DNA in which combination of all the building blocks used has been performed, can be obtained. Specifically, where ligated building block DNA formed by ligation of 2 building blocks, is prepared using n types of building block DNA, ₙC₂ combinations of ligation reaction can be performed at most.

Further, ligation may be performed so as to interleave nucleotide encoding a random 1 to 10, preferably 1 to 5, amino acid residues between the building block DNAs. Such ligated building block DNA can be prepared by PCR using suitable primers.

Further, where an alternative splicing library is to be prepared, ligated building block DNA can be obtained by ligating, in a specific order, 2 building block DNAs selected from building block DNAs produced by segmentation of a single structural gene DNA described in (1) above. A first building block DNA to be selected, can be selected from among the building block DNAs produced by segmentation of a single structural gene DNA described in (1) above, excluding the one present furthermost on the C-terminal side of the protein encoded. Further, a second building block DNA to be ligated to form the ligated building block may be any one as long as it is present further toward the C-terminal side of the protein encoded by the building block DNAs than the above-described first building block DNA. Further, the order of this ligation is such that the 3′-end of the first building block DNA, and the 5′-end of the second building block DNA encoding a building block of the C-terminal side of the encoded protein are ligated, with the amino acid sequences within the building blocks being preserved.

Where ligated building block DNAs are prepared using L building block DNAs segmented from a structural gene where ligated building block DNAs are ligated while preserving their order in the encoded protein from upstream to downstream as described above, ligated building block DNAs are prepared in respect of combinations of all L-1 regions selected as a first building block DNA, and all regions selectable as a second ligated building block DNA. In other words, where a structural gene DNA is segmented into L building block DNAs, _{L}C₂ types of ligated building block DNA are prepared.

(3) Classification of Ligated Building Block DNA

The mutant DNA library according to the present invention can be prepared by performing PCR using the prepared ligated building block as a template. However, where an alternative splicing library is to be prepared, it is necessary to classify the ligated building block DNAs obtained as described above using as an indicator, the number of intervening building blocks that were joined between the above described ligated first building block DNA and second building block DNA in the structural gene in which they were present. Here, X, which is the number of intervening building block DNAs between the ligated first building block DNA and second building block DNA in the structural gene in which they were present, is an integer between 0 and L-2, inclusive, when L is the number of building block DNAs into which the gene was segmented.

Specifically, where ligated building block DNAs are prepared after segmenting DNA ncoding a particular protein into 5 building block DNAs, and the building block DNAs are designated 1, 2, 3, 4, and 5 in order from the 5′-end of the structural gene, 10 types of ligated building block DNA, i.e. 1-2, 1-3, 1-4, 1-5, 2-3, 2-4, 2-5, 3-4, 3-5, 4-5 are prepared. Each of these can be classified as follows: the ones with 0 intervening building blocks between the first building block DNA and the second building block DNA in the structural gene DNA in which they are present (X=0), are 1-2, 2-3, 3-4 and 4-5. Similarly, the ones with 1 intervening building block DNA(X=1), are 1-3, 2-4 and 3-5, 2 intervening building block DNAs (X=2), 1-4 and 2-5, and 3 intervening building block DNAs (X=3), 1-5. Building block DNAs belonging to each of the groups of ligated building block DNAs classified in this manner are generically designated as “ligated building block DNA groups.”

(4) PCR Using Ligated Building Block DNAs as a Template

According to the method of the present invention, by mixing the thus obtained ligated building block DNAs and performing PCR using this as a template, building block DNAs are ligated in arbitrary combinations, and a mutant DNA library consisting of structural genes having various sequences can be constructed in a single PCR.

The ligated building block DNA to be used as a template in this PCR, are selected and mixed such that the end sequences of all the ligated building block DNAs used, overlaps with the end sequences of at least one other type of ligated building block DNA. Here, where a random library is to be prepared, the ligated building block DNA to be used as a template in this PCR, is selected such that all the end sequence of all ligated building block DNAs overlap with the end sequence of at least 1 other type of ligated building block DNA, and either the 5′-end or 3′-end sequence of at least one type of ligated building block DNA overlaps with the end sequence of at least 2 other types of ligated building block DNA. There is no particular limitation on the type and number of ligated building block DNAs to be used in the PCR in this method, as long as they fulfill the above described conditions.

In a random library, where the purpose is to obtain DNAs with all combinations of building block used, PCR may be performed by selecting and mixing ligated building block DNAs formed by ligation of all combinations of building block DNAs to be used, and using this mixture as a template. In this case, it is preferable to use ligated building block DNAs formed by ligation of 2 building block DNAs. Specifically, where a ligation product of n building block DNAs is to be prepared from ligated united region DNAs formed by ligation of 2 building block DNAs using n types of building block DNA, because there will exist n² types of ligated building block DNA with different combinations. By performing PCR using all of these ligated building block DNAs as a template, a mutant DNA library where building block DNAs are ligated with all possible combinations, can be obtained.

In a random library, in respect of the frequency of appearance of a specific building block DNA, this can be regulated by changing the concentrations of the ligated building block DNAs comprising these building block DNAs. Specifically, where a specific building block DNA is expected to appear twice as often as others, ligated building block comprising that building block DNA should be added in an amount twice that of those ligated building blocks which do not comprise the building block DNA.

Further, where an alternative splicing library is to be prepared, ligated building block DNA groups classified as described (3) above, can be used as a PCR template. As the amount of DNA of ligated building block DNA groups to be used in this PCR, the total of each ligated building block DNA group is suitably 300 to 1500 fmol, preferably 500 to 1300 fmol. The amount of each ligated building block DNA group in this template DNA can be calculated respectively according to the following formula (1) relating to X, which is the number of intervening building block DNAs between the first building block DNA and second building block DNA in the structural gene DNA in which they are present; L, which is total number of segmented building block DNAs; and M, which is the difference between the total number of building block DNAs and the number of building block DNAs possessed by the mutant DNA to be prepared:

_{L-X-1}C_{M-X} (1)

wherein, L, X and M have the same meaning as described above.

A value calculated in this manner represents the proportion of the total amount of DNA of each ligated building block DNA group relative to the total amount of the above described template DNA. Further, it is preferable that DNA solution of a certain ligated building block DNA group contains equal amount of molecles for each ligated building block DNA belonging to the group.

Specifically, where the total number of segmented building blocks is 5 (L=5), and the number of building blocks possessed by the mutant DNA that is thought to be prepared is 3 (M=3), the following is the ratio for each ligated building block DNA group to be mixed which are classfied according to the method described above; group X=0, ₄C₂=6; group X=1, ₃C₁=3; group X=2, ₂C₀=1; and group X=0, ₀C₀=0. That is, an amount of DNA being the total amount of 1-2, 2-3, 3-4 and 4-5, which are classified as X=0, mixed in equal amounts, an amount of DNA being the total amount of 1-3, 2-4 and 3-5, which are classified as X=1, mixed in equal amounts, and an amount of DNA being the total amount of 1-4, 2-5, which are classified as X=2, mixed in equal amounts, are mixed such that the proportions thereof are 6:3:1, and PCR is performed with this mixture as a template.

To describe in more detail, for example when the total amount of template DNA is 500 fmol, the amount of DNA being the total of 1-2, 2-3, 3-4, 4-5, classified as X=0, mixed in equal amounts becomes 300 fmol, and 75 fmol each of the above ligated building block DNAs is provided as template DNA for PCR. Similarly with 1-3, 2-4, and 3-5, classified as X=1, equal amounts are mixed to give a total amount of DNA of 150 fmol, i.e. 50 fmol of each of the ligated building block DNA is provided as template DNA for PCR Further, the amount of DNA being the total of 1-4, 2-5, classified as X=2, mixed in equal amounts becomes 50 fmol, i.e. 25 fmol of each ligated building block DNA is provided as template DNA for PCR.

By performing PCR with mixture of suitable amount of ligated building block DNA as template based on the values fulfilling the above described formula (1), a mutant DNA library in which these building block DNAs are ligated by alternative deletion of specific building block DNAs, while maintaining their order in the structural gene from upstream to downstream, can be constructed in a single PCR.

As PCR reaction buffers, and enzymes, etc. suitable known ones can be used. Preferably, where a random library is to be prepared, Vent DNA Polymerase (manufactured by NEB, Inc.), and, where an alternative splicing library is to be prepared, KOD Dash Polymerase (manufactured by Toyobo Co., Ltd..) is used.

The amount of ligated building block DNA to be used in PCR is not particularly limited as long as it is an amount sufficient for a DNA amplification reaction to occur. Specifically, where PCR is performed on a scale of a 50 μl reaction, a suitable total amount would be 300 to 1500 fmol, preferably 500 to 1300 fmol.

Further, any PCR conditions may be used as long as a DNA amplification reaction occurs with the ligated building block DNA as a template. Preferable conditions can be suitably selected, adapted, and used based on the length of the overlapping sequence of each ligated building block DNA and the length of building block DNA, or the length of the ligated DNA of ligated building block that is to be finally constructed, etc., in accordance with the descriptions on p. 13-43 of Bio Jikken Illustrated 3, Saibou Kougaku supplement, Hiroki Nakayama, Shujunsha (1993), Chapter 1. Specifically, suitable conditions include performing a DNA denaturation (denature) reaction at 94 to 96° C., preferably 95° C., for 15 to 60 seconds, preferably 20 to 40 seconds, performing primer binding (annealing) at 50 to 70° C., preferably 55 to 65° C., for 15 to 60 seconds, preferably 20 to 40 seconds, and then performing polymerase elongation (elongation) reaction, at 70 to 74° C., preferably 72 to 73° C. for 1 to 5 minutes, preferably 2 to 3 minutes as 1cycle, and repeating 15 to 40 cycles, preferably 20 to 30 cycles.

Primers are not essential in the above described PCR. However, PCR can be performed using a suitable primer. Advantages of performing PCR using primers include the fact that because DNAs having both ends with sequences complementary to the primers, are selectively amplified, it is possible to selectively obtain DNAs having any building block or a building block comprising a common sequence at both ends of the constructed DNA, and that because the amount of the constructed DNA increases it is easier to identify the PCR reaction product during isolation, etc. As an amount of primer, 10 to 60 pmol, preferably 20 to 40 pmol, of each of forward and reverse primer is suitable.

Where a mutant DNA library is to be constructed where building block DNAs to form both ends of the DNAs to be constructed are fixed, it is preferable to perform PCR in the presence of primers having sequences complementary to the building block DNAs which are to form both ends of the DNA to be constructed.

By performing PCR as described above, a DNA library comprising 2 or more clones can be obtained. A “DNA library comprising 2 or more clones” refers to a library consisting of 2 or more clones having differing DNA sequences included in the obtained library.

By adding an appropriate manganese ion in this PCR buffer, nucleotide substitution can be induced at a specific frequency in each DNA sequence at the preparation of the DNA library of the present invention. Specific sources of manganese ions include manganese chloride, etc. The manganese ion concentration in PCR buffer is selected according to the frequency of nucleotide substitutions to be induced. The relation between nucleotide substitution frequency and manganese ion concentration can be determined by performing PCRs with altered manganes ion concentration, analyzing the nucleotide sequences of the DNA fragments contained in the obtained DNA libraries, and examining the rate of nucleotide substitution induced in the DNAs. As this function, the values indicated in Table 2 of Example 3, for example, can be used. As another method, nucleotide substitution can be induced in the DNA sequences contained in the prepared mutant DNA library by altering the dNTP composition.

(5) PCR using Ligated Building Block DNAs having Common DNA Sequences, as Templates

A preferable embodiment of the above described PCR using ligated building block DNAs as templates, is a method involving performing PCR using ligated building block DNAs such that different common sequences are added respectively to both ends of the DNA to be constructed, as templates, and using primers having sequences complementary to the common DNA sequences. By this method, a library of mutant DNAs having common DNA sequences at both ends can be selectively constructed and obtained.

A common DNA sequence may be an arbitrary sequence having a length of about 15 to 100 base pairs. However, where the constructed DNA is to be used in the below described preparation of mutant protein library, it is preferred that the 5′-end common sequence is a suitable promoter sequence.

There is no particular limitation on the promoter sequence that can be used as a common DNA sequence as long as it enables expression of the protein encoded by the DNA in the host for expressing the DNA. Specifically, where the host organism is E. coli, T3, T7, SP6, tac, or lac promoter, etc. can be used, and in the case of yeast, nmtl promoter or Gal1 promoter, etc. can be used. Further, in the case of cultured animal cells, SV40 promoter or CMV promoter, etc. can be used.

Preparation of ligated building block DNA having a common DNA sequence can be performed by preparing a 2-stranded DNA having a common sequence as 1 building block DNA according to the method of preparing a building block DNA described in (1) above, and ligating this according to the method of preparing a ligated building block DNA described in (2) above. Specifically, a desired 5′-end common DNA sequence and a building block DNA, and a 3′-end common DNA sequence and a building block DNA are ligated by similar operations to the above described preparation of ligated building block DNA. Next, the ligation product of the 5′-end common DNA sequence and building block DNA, and the ligation product of the 3′-end common DNA sequence and building block DNA, can be prepared by performing PCR using the 5′-side common DNA sequence as a forward primer, and the 3′-end gene sequence of the building block DNA ligated thereto as a reverse primer; and for the 3′-end side, the 3′-side common DNA sequence as a reverse primer, and the 5′-end DNA sequence of the building block DNA ligated thereto as a forward primer.

Further, the addition can be attained also by performing PCR for the construction of ligated building block DNA described above using primers which encode the sequence complementary to the end sequence of building block DNAs and are ligated to the common sequence at 5′- or 3′-end.

Ligated building block DNAs comprising a common DNA sequence prepared in this manner are mixed with other ligated building block DNAs, and PCR is performed. The method of selecting and the mixing ration of other ligated building block DNAs can be performed based on the description in (4) above. The details are described below.

In a random library, among the mutant DNA libraries prepared by the method of the present invention, where the object is to obtain mutant DNAs having a common sequence at the 5′-end or 3′-end and having all combinations of building block DNA to be used in recombination, ligated building block DNAs formed by ligation of the building block DNAs with all possible combinations, and ligated building block DNAs formed by addition of a common sequence to the 5′-end or 3′-end of all building block DNAs which are used, are selected, mixed, and PCR can be performed with this mixture as a template. In this case, it is preferable to use the ligated building block DNA formed by ligation of 2 building block DNAs, Specifically, where ligatied product DNAs of n number of building block DNAs having a common sequence at the 5′-end or the 3′-end is prepared by PCR using ligated building block DNAs formed by ligation of 2 building blocks from n types of building block DNA, n² ligated building block DNAs and 2n ligated building block DNAs having a common sequence at the 5′-end or the 3′-end are mixed and PCR is performed with this mixture as a template, a random DNA library in which all combinations of building block DNAs used are ligated, and which has a common sequence at the 5′-end or the 3′-end, can be obtained.

Further, in the alternative splicing library, ligated building block DNAs comprising a common DNA sequence are mixed at a specific ratio and by performing PCR with this mixture as a template, the library can be constructed by a single PCR.

The mixing proportion of the obtained ligated building block DNAs formed by ligation of a common DNA sequence and a building block DNA, can be calculated in the manner described in (3) above based on the number of intervening building block DNAs between the common DNA sequence and the building block ligated thereto, in the structural gene in which they are present (X: however, X is an integer between 0 and L-1, inclusive, when L is the number of segmented building blocks), and this is mixed with other ligated building block DNAs, and PCR is performed.

Specifically, where ligated building block DNAs are prepared after segmenting DNA encoding a protein into 5 building block DNAs, the building block DNAs are designated as 1, 2, 3, 4, and 5 in the order from 5′-side of the structural gene, the 5′-common DNA sequence is designated as T, and the 3′-common DNA sequence is designated as C. Then 10 types of ligated building block DNAs formed by ligation of the common DNA sequence T or C and building block DNAs, T-1, T-2, T-3, T-4, T-5, 1-C, 2-C, 3-C, 4-C and 5-C, are prepared. Of these, those with 0 intervening building block DNAs between the common DNA sequence and the building block DNA in the structural gene DNA in which they are present (X=0) are T-1 and 5-C. Similarly, those with 1 intervening building block (X=1) are T-2 and C-4. Those with 2 intervening building blocks (X=2) are T-3 and 3-C. Those with 3 intervening building blocks (X=3) are T-4 and 2-C, and those with 4 intervening building blocks (X=4) are T-5, 1-C. Consequently, the mixing ratio for each ligated building block DNA to be used in PCR, can be calculated from each X value.

In the PCR using, ligated building block DNA groups comprising ligated building block DNAs having common sequences selected and mixed in this manner, use of primers having a sequence complementary to the common DNA sequence added to the 5′-end or the 3′-end are required.

PCR can be performed using reaction buffers, enzymes, and reaction conditions, etc., based on the method described in (4) above. However, in respect of ligated building block DNAs comprising a common sequence, more preferably, where PCR is performed on a scale of a 50 μl reaction, a total amount of 5 to 30 fmol, preferably 10 to 20 fmol is used.

(6) Principle of the Method of Constructing an Alternative Splicing Library

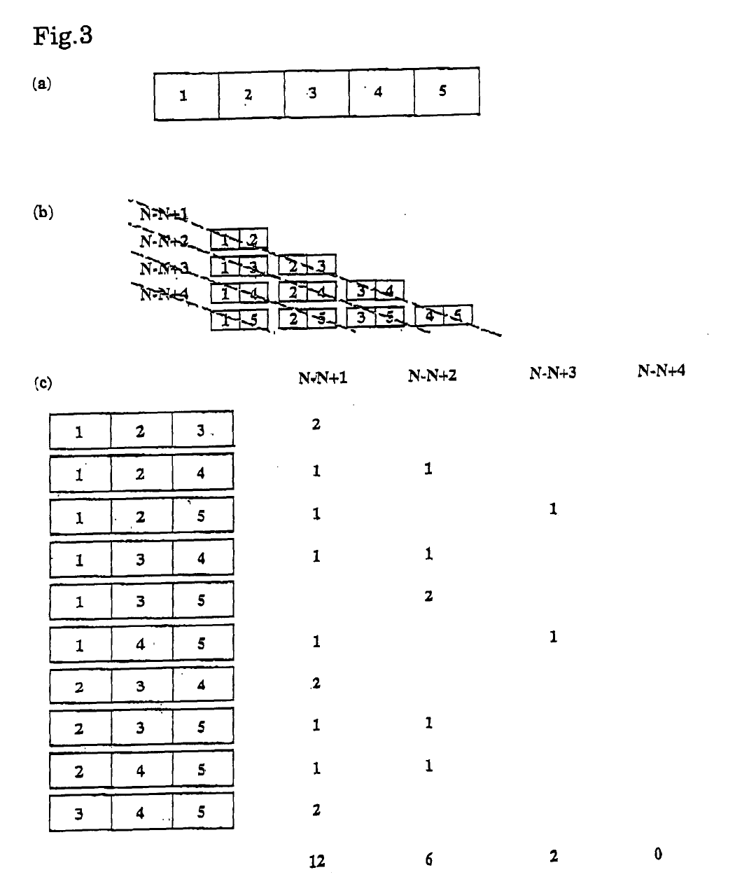

Of the mutant DNA libraries according to the present invention, an example of the protein composed of 5 building blocks shown in FIG. 3 is explained in connection with the principle of the method of constructing an alternative splicing library. In this case, ₅C₂=10 types of ligated building block DNA formed by ligation of these two while preserving the order in the structural gene from upstream to downstream, can be contemplated. These ligated building block DNAs can be classified in 4 classes (FIG. 3b) according to the building blocks which they encode. Where the mutant protein to be constructed by ligation with alternative deletion of arbital building blocks is a protein comprising 3 building blocks selected from 5 building blocks, ₅C₃ 10 structures of this mutant protein can be contemplated (FIG. 3c).

Next, the ligated building blocks identified within these 10 types of sequence indicated above, are counted, and these are classified based on FIG. 3b. As a result, where the building blocks are designated N, N+1, N+2, N+3, N+4 from the N-terminal side of the encoded protein, there are 12 N−N+1 (number of intervening building blocks (X)=0) ligated building blocks, 6 N−N+2 ligated building blocks, 2 N−N+3 ligated building blocks, and 0 N−N+4 ligated building blocks.

With reference to this value, in order to efficiently produce a DNA library encoding a group of proteins wherein from a protein composed of 5 building blocks, an arbitrary 2 building blocks are deleted (in the case of the present Example, 10 types), it is clear that PCR should be performed using ligated building block DNA mixed in the ratio of N−N+1(number of intervening building blocks (X)=0):N−N+2:N−N+3:N−N+4=6:3:1:0, as a template.

Where the above described is expanded to a general formula, where a structural gene DNA encoding a protein is divided into L building block DNAs (where L is any integer), and a ligated mutant DNA library is to be prepared by PCR wherein, of these, M building blocks, are selectively deleted, the proportion of ligated building block DNA to be used as a template can be calculated according to the following formula (1) when X is the number of intervening building blocks (X) in the structural gene between 2 of the above described building block DNAs that are ligated while preserving their order in the structural gene from upstream to downstream to form the ligated building block DNA:

_{L-X-1}C_{M-X} (1)

(7) Purification of Mutant DNA Library

DNA fragment contained in a DNA library constructed by amplification by PCR as described in (4) or (5) above can be confirmed by electrophoresis, etc. using agarose gel, etc.

Further, from a mixture of DNA formed by building blocks ligated in various numbers, a mutant DNA library composed of DNA of an arbitrary specific length, or a mutant DNA library in which DNA formed by ligation of the target number of building block DNAs are most numerous can be obtained by electrophoresis using the above described agarose gel, etc. In this case, it is preferable to use low melting point agarose gel. DNA is subjected to electrophoresis in the gel, a portion comprising DNA of any specific length is excised, and the DNA can be extracted and purified from the excised gel by using known methods.

The thus obtained mutant DNA library can be used as a DNA library or RNA library to analyze protein-nucleic acid interaction. Further, this can be used for preparation of a protein library encoded by the DNA contained in the DNA library (hereinafter, also called a “mutant protein library”) by introduction into a suitable vector, and transformation of a suitable host with the recombinant vector, or by a method of expressing in a cell free translation system after transcription, etc.

(8) Introduction of the Mutant DNA Library into a Vector, and Preparation of a Mutant Protein Library

There is no particular limitation on the vector that can be used in the preparation of the recombinant vector, as long as it enables the expression of the DNA contained in the mutant DNA library according to the present invention in a host organism or cultured cells. However, a commercially available vector for protein expression having inserted thereinto a promoter suitable for a host organism or cultured cells, can be used. As described above, where ligated building block DNAs having a suitable promoter sequence added to the 5′-end thereof as a common DNA sequence are used as a template for PCR when constructing the mutant DNA library, ordinary cloning vectors can be used. Further, even in the case of a mutant DNA library in which the above described promoter sequence DNA has not been added to the 5′-end, if a suitable promoter sequence DNA is added by ligation to the 5′-end thereof, an ordinary cloning vector can be used.

Examples of protein expression vectors include, where the host organism is E. coli, pET3, pET11 (Stratagene), and pGEX (manufactured by Amersham Pharmacia Biotech), etc., or where the host is yeast, pESP-I expression vector (Stratagene), etc. Where cultured animal cells are used as a host, ZAP Express (Stratagene) and pSVK3 (manufactured by Amersham Pharmacia Biotech), etc., can be used. Where insect cells are used, BacPAK6 (manufactured by Clontech), etc. can be used.

As a promoter for expression of the DNA contained in the mutant DNA library according to the present invention, typically a promoter possessed by the host organism or cultured cells can generally be used but it is not limited to this. Specifically, where the host organism is E. coli, T3, T7, SP6, tac, or lac promoter can be used. Further, where yeast is used as a host, nmtl promoter or Gall promoter, etc. can be used, Further, in the case of cultured animal cells, SV40 or CMV promoter, etc. can be used.

Insertion of the DNA contained in the mutant DNA library according to the present invention into these vectors, can be performed by ligation using DNA ligase, etc, such that the DNA fragment is present downstream of the above described promoter in the vector.

A transformant can be prepared by introduction of the thus obtained recombinant vector into a suitable host, by a suitable known method. Specific examples of methods of introduction into a host, include heat shock method (J. Mol. Biol., 53, 154-(1970)), calcium phosphate method (Science,221,551-(1983)), DEAE dextran method (Science,215,166-(1982)), electric pulse method (Proc. Natl. Acad. Sci. USA, 81, 7161 (1984)), in vitro packaging (Proc. Nati. Acad. Sci. USA, 72, 581 (1975)), and virus vector method (Cell, 37, 1053 (1984)).

At this time, any host may be used as a host for introduction of the DNA as long as it enables expression of the protein encoded by the DNA within the host by the recombinant vector for the protein expression comprising the DNA. Examples of hosts include E. coli, yeast, baculovirus (anthropod polyhedral virus)-insect cells, animal cells and cultured animal cells.

Specific examples include BL21, XL-1-Blue (Stratagene), etc. for E. coli, SP-Q01(Stratagene) for yeast, and AcNPV (J. Biol. Chem., 263, 7406 (1988)) and its host, Sf-9 (J. Biol. Chem., 263,7406(1988)) for baculovirus. Further, examples of cultured animal cells include mouse fibroblasts C127 (J. Viol., 26, 291 (1978)), Chinese hamster ovaries CHO (Proc. Natl. Acad. Sci. USA., 77, 4216 (1980)) and African Green Monkey renal cells, COS (ATCC: CRL1651).

Expression of the proteins encoded by the DNA of the mutant DNA library can be induced by cultivation of the thus obtained transformants by using methods respectively suitable therefor, on suitable media. The medium used for cultivation comprises a carbon source, nitrogen source, inorganic matter, vitamins, serum, necessary for development of the transformant, and drugs, etc. to be used in resistance screening. Specifically, where the transformant host is E. coli, for example, LB medium (Nacalai Tesque, Inc.), etc., or where the host is yeast, YPD medium (Genetic Engineering, 1, 117, Plenum Press(1979)), etc., and in the case of insect cells, animal cells, and cultured animal cells, MEM medium, DMEM medium, RPMI1640 medium (Sigma) comprising 20% or less fetal calf serum, etc. can be used.

Cultivation of transformants is typically performed at a temperature range of 20 to 45° C. and a pH range of 5 to 8, and where necessary airation and agitation are conducted. There is no particular limitation on the medium composition and culture conditions other than the above, as long as the transformants grow and the protein encoded by the DNA that was introduced is produced.

Cells or bacteria are collected from the obtained culture by methods such as centrifugation, suspended in a suitable buffer, and disrupted by suitable known methods such as ultrasonication, lysozyme and/or freeze-thawing. Thereafter, a crude protein solution is obtained by centrifugation or filtration, etc. and a purified protein can be further obtained by a combination of suitable purification methods. Thus, the mutant protein library is prepared.

(9) Expression of the Mutant DNA Library by a Cell Free Translation System, and Preparation of a Mutant Protein Library

In addition to the above described method of expression using a protein expression recombinant vector, a mutant protein library can be prepared by inducing protein expression by subjecting an RNA library obtained by transcription of the mutant DNA library into a cell free translation system. Transcription of the DNA library can be by any typically used known method, however, transcription can also be performed simultaneously with translation using a cell free transcription/translation system. The cell free transcription/translation system usable in the present invention indicates any system comprising all elements required for transcription from DNA to mRNA and translation from MRNA to protein, wherein, by addition of DNA thereto, protein encoded by that DNA is synthesized.

Specific examples of a cell free transcription/translation system include transcription/translation systems prepared from an extract solution of eukaryotic cells, bacteria cells, or a portion thereof, and particularly preferable examples include transcription/translation systems prepared from extract solutions of rabbit reticulocytes, wheat germ or E. coli (E. coli S30 extract), etc.

Separation and purification of the proteins encoded by the mutant DNA library from the obtained transcription/translation products of the cell free transcription/translation system can be performed by any typically used known method. Specifically, for example, a DNA region encoding epitope peptide, polyhistidine peptide, glutathione-S-transferase (GST), maltose binding protein, etc. can be introduced into the above-mentioned DNA to be transcribed/translated, and the protein is expressed as described above, and purified by using affinity between said protein and a substance having affinity thereto.

(10) Screening of the Mutant Protein Library

The mutant protein library obtained as described in (8) or (9) above, can be applied for screening using possession of a desired property as an indicator. A protein having a desired property refers to a protein binding to a desired target molecule, a protein catalyzing a desired chemical reaction, or the above described protein having altered binding ability, catalytic ability, substrate specificity, or stability against heat and/or environmental conditions.

Methods of selecting a protein having a desired property using target biological activity include a method of selection using binding ability to a target molecule as an indicator, and a method of selection using catalytic activity as an indicator. A protein having a desired property can be selected using these binding abilities or catalytic activities as an indicator by using such methods as, for example, affinity resin adsorption, immunoprecipitation, electrophoresis, chromatography, or flow cytometry. More specifically, a target molecule such as a protein, nucleic acid (DNA or RNA), saccharide, or lipid, etc. is first bound to a solid phase such as a microplate or beads, and the mutant protein library constructed as described above is added thereto, and after allowing reaction under suitable temperature conditions for certain period, washing is performed, and the mutant proteins bound to the target molecule are selected, This method can be performed according to the already established Enzyme Ligated Immunosorbent Assay (ELISA) (Crowther, J. R., 1995, Method in Molecular Biology, Vol. 42, Humana Press Inc.).

When selecting a protein having a desired property using catalytic activity as an indicator, for example, an enzyme protein, the following 2 methods, for example, can be used. One is a method, as in the case of a catalytic antibody, of selecting a protein binding to a resin displaying a transition state analog of a desired catalytic reaction (Tramontano, A. et al, (1986) Science, 234, 1566-1570). Another is a method, as in the case of RNA enzyme (ribozyme), of designing selection conditions such that proteins in the mutant library bind to or dissociate from the resin by the formation or cleavage of the bond between molecules which is arisen from the catalytic activity (Gold, L. et al. (1995) Annu. Rev. Biochem., 64, 763-797). Whereas with the former selection method it is difficult to increase turnover of enzyme activity, the latter method is from the start a method of selection based on the catalytic activity not binding, as can be seen from the many examples of success with ribozymes, and thus the latter method is more effective.

Further, the fact that the structure of catalytic antibodies is restricted as the immunoglobulin fold is also one reason why products having as much activity as natural enzymes are not being obtained, In the present invention, a ribozyme-type selection method can be employed, so the selection range of protein structure is free and not limited to structures of antibodies, and it is expected that it will contribute greatly to the creation of novel catalytic proteins exceeding the conventional catalytic antibody.

(11) Obtaining a Protein having an Improved Desired Property

A new mutant protein library can be prepared by preparing the DNA encoding a protein selected by the screening method described in (10), introducing mutations into the DNA, amplifying the DNA having mutations introduced thereinto, and expressing the amplified DNA. A protein wherein the desired property is improved can be obtained by the selection of a protein having the desired property as an indicator using this new library. Further, a protein in which the desired property is further improved can also be obtained by repeating the above operation with the protein obtained in this manner. As a method for preparing the DNA encoding a protein selected by using possession of a desired property as an indicator, a method which involves recovering the DNA from transformants producing this protein by known methods, and amplifying using PCR, etc., can be used. For amplification of DNA encoding a desired protein by PCR, primers having a sequence complementary to the sequence of the vector into which the mutant DNA was inserted, or in the case ligated building block DNAs having a common DNA sequence were used, primers having a sequence complementary to the common DNA sequence, will be used.

Further, as methods of inducing mutations in the amplified DNA, the already established methods of Error-prone PCR (Leung, D. W. et al.(1989) J. Methods Cell. Mol. Biol., 1, 11-15) or Sexual PCR (Stemmer, W. P. C. (1994) Proc. Natl. Acad. Sci. USA, 91, 10747-10751) can be used.

The thus obtained protein in which the desired property is further improved can be obtained by isolation and purification according to the known methods.

(12) Obtaining a Protein having an Improved Desired Property by Using the Nucleic Acid-protein Ligated Molecule

The method of obtaining a protein in which the desired property is improved according to (11) above, can be even more efficiently performed by the use of a nucleic acid-protein ligated molecule formed by ligation of the protein with a nucleic acid encoding the protein in a corresponding manner. A nucleic acid-protein ligated molecule can be prepared according to methods such as phage display method (Smith, G. P. (1985) Science 228, 1315-1317), ribosome display method (Hanes, J. and Pluckthun, A. (1997) Proc. Natl. Acad. Sci. USA 94, 4937-4942), in vitro virus method (Nemoto, N. et al. (1997) FEBS Lett. 414, 405-408; Roberts, R. W. and Szostak, J. W.(1997) Proc. Natl. Acad. Sci. USA 94, 12297-12302; International Publication No. W098/16636), and STABLE (Doi, N., et al. (1999) FEBS Lett. 457, 227-230), etc.

Phage Display is a method using a phage that infects and propagates in E. coli. Phage particles take the form of coat proteins constituting the phage which envelopes the DNA which encodes it. By inserting DNA into the coat protein gene of a phage and allowing it to express, a library of phages which present peptides encoded by DNA on their surface can be prepared. That is, though indirectly, the protein which is present on the surface of the phage particle is linked to the DNA present in the interior of the phage. DNA encoding a presented peptide which is selected from among the peptides being presented by using the possession of a desired property as an indicator, can be obtained by recovering the DNA from the interior of the selected phage.

In Ribosome Display, when translating mRNA whose stop codon has been removed, by a cell free translation system, etc., the synthesized protein is trapped on the ribosome. As a result, a protein-ribosome MRNA complex is prepared, which can be used as a protein-nucleic acid ligated molecule. DNA encoding a protein selected from among proteins trapped on the ribosomes using possession of a desired property as an indicator, can be obtained from the ribosome bound to the selected protein.

In the in vitro virus method, when mRNA is expressed using a cell free protein synthesis system, etc., an mRNA-protein ligated molecule in which the mRNA and protein are chemically bound can be constructed on a ribosome. Further, this mRNA-protein ligated molecule (in vitro virus) can be subjected to selection with an in vitro selection method, and DNA can be amplified and obtained by RT-PCR using mRNA portion of the in vitro virus selected.

At STABLE, a ligand substance such as biotin has been bound to DNA previously, the adaptor protein such as streptavidin which binds to the ligand is expresses as fusion protein with the protein encoded by the DNA, and the protein and nucleic acid can be linked through the bond between the ligand and adapter protein. Specifically, for example, biotin is first chemically bound to the end of a DNA, and the DNA is expressed in a cell free transcription/translation system. At this time, 1 molecule of DNA is allowed to express in a separated state within a microcapsule, etc. The synthesized protein will bind to the DNA encoding the protein through the bond between the ligand, biotin, linked to the DNA in the microcapsule, and the adapter protein, streptavidin, expressed as a fusion protein, resulting in the formation of a protein-DNA ligated molecule. DNA encoding a protein selected by using as an indicator possession of a desired property by the protein portion of the ligat d molecule, can be obtained by amplifying the DNA protein of the select d ligated molecule by PCR.

As in the (11) above, a new mutant protein library can also be prepared by introducing mutations into the DNA encoding a protein having a desired property obtained using a nucleic acid-protein ligated molecule such as this, amplifying the DNA having mutations introduced thereinto, and expressing the amplified DNA. A protein wherein the desired property is improved can be obtained by selection of a protein using the desired property as an indicator using the new library. Further, a protein in which the desired property is further improved can also be obtained by repeating the above operation using the protein obtained in this manner. The thus obtained protein in which the desired property is further improved can be obtained by isolation and purification according to the known methods described above.

Screening of the protein having a desired property may be performed in a manner similar to the method described in (10) above.

(13) Obtaining a DNA Encoding the Protein having a Desired Property

The recombinant protein having a desired property according to (10), (11) or (12) above can be prepared by analyzing the protein and obtaining the DNA that encodes it.

DNA encoding a protein, in the case of a protein obtained by the screening method according to (10) or (11) above, can be obtained by analysis using a suitable combination of typically used known methods. Specifically, in the case of a protein obtained from an alternative splicing library, for example, (1) the molecular weight of the protein can be determined using a mass spectrometer, etc., (2) and the protein can be identified from this molecular weight by calculating the type and number of building blocks included in the protein. A DNA fragment having an identified DNA sequence can be prepared in a manner similar to the method of preparing ligated building block DNA described in (2) above.

Further, in the case of a protein obtained from a random library, for example, the amino acid sequence of the protein can be analyzed by publicly known methods, and the type and order of linking of building blocks contained can be identified from amino acid sequences obtained. A DNA fragment having an identified DNA sequence can be prepared in a manner similar to the method of preparing ligated building block DNA described in (2) above.

Further, in the case of a protein obtained by the screening method described in (12) above, the nucleic acid portion of the obtained nucleic acid-protein ligated molecule can be obtained by amplification by reverse-transcription PCR or PCR.

Further, herein, operations of isolation, preparation, ligation and synthesis of DNA, or methods of transformation, construction of plasmids, cultivation of transformants and the like can be performed according to methods described in or adapted from methods described in Sonmbrook, J. et al. (1989) Molecular Cloning, 2^{nd} Edition, Cold Spring Harbor Laboratory Press, except as where otherwise stated.

The entire contents of the specifications of Japanese Patent Applications Nos. 2000-293692 and 2001-29138 which form the basis of priorities which the present application claims, are incorporated herein by reference as part of the disclosure of the present specification,.

Below, the present invention is explained in further detail by means of Examples. However, the Examples below should be considered as aides to obtaining a concrete understanding of the present invention, and they do not limit scope of the present invention in any way.

### EXAMPLES

### Example 1

(1) Preparation of the Building Block DNA

The whole region of the dinucleotide binding site and a part of the acceptor domain of glutaminyl-tRNA synthetase (Rould, M. A. et al. (1989) Science 246, 1135-1142) is composed of 150 amino acid residues (Hoben, P. et al. (1982) J. Biol. Chem., 257, 11644-11650). The whole region of this dinucleotide binding site and a region corresponding to. a part of the acceptor domain were divided into 6 equal parts consisting of 75 base pairs, encoding 25 amino acid residues, to be used as building block DNA (1 to 6; SEQ ID NO: 1 to 6). The building block DNA fragments were prepared by chemical synthesis (Espec Oligo Service Corporation). Further, as a 5′-common sequence, a DNA fragment comprising T7 promoter sequence (T7; SEQ ID NO: 19) was prepared by chemical synthesis (Espec Oligo Service Corporation). Similarly, a 3′-common sequence (Ex; SEQ ID NO: 20) was prepared by chemical synthesis (Espec Oligo Service Corporation).

Each of the building block synthetic DNA fragments, and the common sequence synthetic DNA fragments were made into double strands by annealing, and then amplified using forward primers (F1 to F6; SEQ ID NO: 7 to 12) having homology to the 5′-side nucleotide sequence of the building block DNAs and reverse primers (R1 to R6; SEQ ID NO: 8 to 18) having homology to the 3′-side nucleotide sequence, and forward primers (FT7; SEQ ID NO: 21, FEx; SEQ ID NO: 23) having homology to the 5′-side nucleotide sequence of each of the common sequences, and reverse primers (RT7; SEQ ID NO: 22, REx; SEQ ID NO: 24)(Dateconcept) having homology to the 3′-side nucleotide sequences.

Each primer was phosphorylated prior to PCR. The phosphorylation reaction solution (30 μl ) comprised 20 μl of DNA solution, 10 mM of ATP, 1 unit of polynucleotide phosphate kinase (manufactured by New England Biolabs, Inc.), and 3 μl of the provided 10× reaction buffer. The PCR reaction solution (50 μl) comprises 20 pmol of each primer, 200 μM of dNTPs, 1 unit vent DNA polymerase (manufactured by New England Biolabs, Inc.), and 5 μl of the provided 10×vent DNA polymerase buffer. The reaction was performed with the following reaction conditions: Stage 1 (1cycle), 95° C. for 5 minutes; Stage 2 (30 cycles), 95° C. for 30 seconds, 60° C. for 30 Stage 3 (1cycle), 4° C. for 10 minutes. A total of 8 types of DNA fragment, that is, the 6 building block DNA fragments amplified by PCR, 5′-common sequence DNA fragment, and the 3′-common sequence DNA fragment, were extracted using phenol and chloroform, precipitated with ethanol, and dissolved in 50 μl of TE buffer.

(2) Preparation of the Ligated Building Block DNA

The 8 types of DNA fragment obtained by amplification in (1) described above, were ligated in the following combinations:

T7-1, T7-2, T7-3, T7-4, T7-5, T7-6, 1-2, 1-3, 1-4, 1-5, 1-6, 1-Ex, 2-3, 2-4, 2-5, 2-6, 2-Ex, 3-4, 3-5, 3-6, 3-Ex, 4-5, 4-6, 4-Ex, 5-6, 5-Ex, 6-Ex.

The reaction solution comprises 10 μl of DNA solution and 10 μl of Ligation High (manufactured by Toyobo Co., Ltd.). The reaction was conducted at 16° C. for 1 hour. The solution containing ligated building block DNA in which this reaction was performed, was named “ligation solution”.

Using the above described ligation products as the template DNA, each ligated building block DNA formed by ligation of 2 building block DNAs, were amplified using the suitable primers used in (1) above. The following 48 types of the ligated region DNA were amplified: T7-1, T7-2, T7-3, T7-4, T7-5, T7-6, 1-1, 1-2, 1-3, 1-4, 1-5, 1-6, 1-Ex, 2-1, 2-2, 2-3, 2-4, 2-5, 2-6, 2-Ex, 3-1, 3-2, 3-3, 3-4, 3-5, 3-6, 3-Ex, 4-1, 4-2, 4-3, 44, 4-5, 4-6, 4-Ex, 5-1, 5-2, 5-3, 5-4, 5-5, 5-6, 5-Ex, 6-1, 6-2, 6-3, 6-4, 6-5, 6-6, and 6-Ex.

The PCR mixture (50 μl) comprises, 20 pmol of each primer, 200 μM of dNTPs, 1 μl of ligation solution, 1.5 mM MgCl₂, 1 unit Taq DNA polymerase (manufactured by Toyobo Co., Ltd..), and the provided 10×Taq DNA polymerase buffer. PCR was performed with the following reaction conditions: Stage 1 (1 cycle), 95° C. for 5 minutes; Stage 2 (30 cycles), 95° C. for 30 seconds, 60° C. for 30 seconds; Stage 3 (1 cycle), 4° C. for 10 minutes. After ethanol precipitation and dissolution in 20 μl of sterilized water, these PCR reaction solutions were subjected to electrophoresis using low melting point agarose gel. After electrophoresis, the target DNA band was excised, and after extracting with phenol and chloroform, precipitated with ethanol, and dissolved in 60 μl of sterilized water. The concentrations of the ligated building block DNAs were determined by measuring adsorption at 260 nm. The concentrations of all ligated building block DNAs were around 60 to 100 ng/μl.

The obtained 48 types of ligated building block DNA were cloned using TOPO TA Cloning KIT (manufactured by Invitrogen). Cloning procedures were as according to the protocol provided with the KIT (TOPO TA Cloning Ver. K: Five Minutes Cloning of Taq Polymerase-Amplified PCR Products, Invitrogen). First, 4 μl of the ligated building block DNA PCR product was mixed with 1 μl of the salt solution provided with the KIT and 1 μl of PCR-TOPO vector, and incubated at 25° C. for 5 minutes. Next, 2 μl of the reaction solution was mixed with 50 μl of competent cells provided with the KIT, and this was incubated on ice for 30 minutes. Thereafter, after incubating at 42° C. for 30 seconds, 250 μl of SOC medium that was provided with the kit was added to all of competent cells, and the cells were then cultured at 37° C. for 30 minutes. Thereafter, 50 μl of the SOC was applied to an LB plate having 50 μg/ml ampicillin added thereto. LB plates to which 24 μl of 40 μg/ml isopropyl-1-thio-β-D-galactoside galactose (IPTG) and 40 μl of 40 μg g/ml X-gal solution had first been applied, were used.

After incubating the above described LB plate for 12 hours at 37° C., around 200 colonies were confirmed on the plate. Three blue colonies for each ligated building block DNA were selected, and whether or not the target ligated unit DNA was contained in the plasmid, was examined by colony PCR. The reason why blue not white colonies were selected was because all of the building block DNAs and the common sequence DNAs consisted of a number of base pairs being a multiple of 3, and it was predicted that this would not cause a frame shift in lacZ gene and have no influence on activity. The colony PCR mixture (50 μl) comprises 20 pmol of each primer, 200 μM of dNTP, 1.5 mM MgCl₂, 1 unit Taq DNA polymerase (manufactured by Toyobo Co., Ltd..), and 5 μl of the provided 10×Taq DNA polymerase buffer. As template DNA, a colony suspended in sterilized water was used. Reaction conditions were as follows: Stage 1 (1cycle), 95° C. for 5 minutes; Stage 2 (30 cycles), 95° C. for 30 seconds, 60° C. for 30 seconds, 72° C. for 30 seconds; Stage 3 (1 cycle), 4° C. for 10 minutes. Colony PCR products were analyzed by agarose electrophoresis, and after confirming amplification of the target ligated building block DNAs, the products were purified using Wizard PCR Preps DNA Purification System (manufactured by Promega), and analysis of the nucleotide sequence.was performed using a DNA Sequencer (CEQ2000 DNA Analysis System; manufactured by Beckman Coulter, Inc.). By DNA sequencing, the absence of mutations such as substitutions, insertions and deletions in the prepared ligated building block DNAs was confirmed, and these were used as template DNAs in the next PCR.

(3) Preparation of a Mutant DNA Library

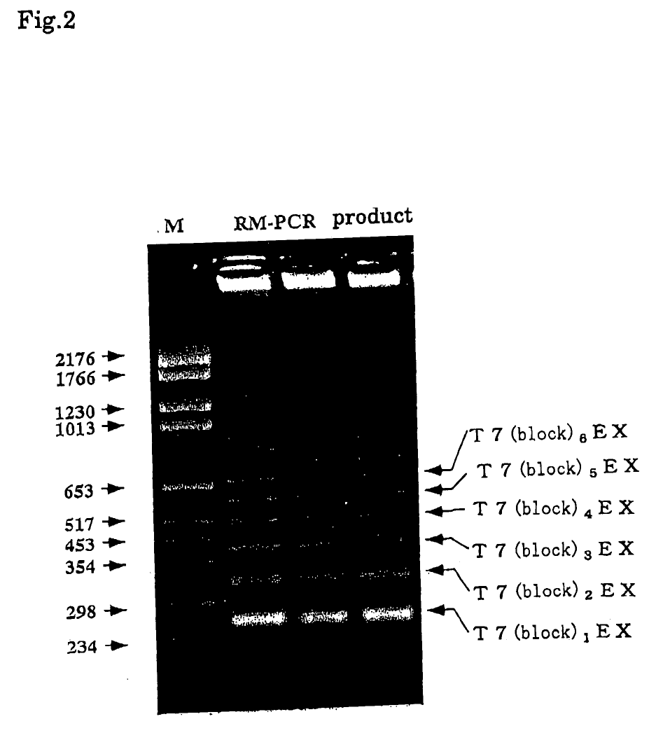

All of the ligated building block DNAs prepared in (2) above were mixed and PCR was performed. The PCR mixture (50 μl) comprises, as primers, 20 pmol each of T7 forward primer (SEQ ID NO: 21) and Ex reverse primer (SEQ ID NO: 24), a total of 750 fmol of the 36 types of ligated building block DNA comprising building blocks 1 to 6, and a total of 15 fmol of 12 types of ligated building block DNA comprising T7 and Ex, 200 μM of dNTPs, 1 unit of Vent DNA polymerase (manufactured by New England Biolabs, Inc.), and 5 μl of the provided 10×vent DNA polymerase buffer. Reaction conditions were as follows: Stage 1 (1 cycle), 95° C. for 5 minutes; Stage 2 (20cycles), 95° C. for 30 seconds, 60° C. for 30 seconds, 72° C. for 3 minutes; Stage 3 (1cycle), 4° C. for 10 minutes. After concentrating the obtained PCR product by ethanol precipitation, 15 or more bands in ladder form could be confirmed by electrophoresis using low melting point agarose gel (FIG. 2). Because the ladder intervals corresponded to 75 base pairs, this ladder was predicted to correspond to the sequences T7-(X)-Ex, T7-(X)²-Ex, T7-(X)³-Ex, . . . , T7-(X)¹⁵-Ex, . . . in order from the smallest molecular weight (“X” is any one of building blocks 1 to 6). That is, the possibility that the 6 building blocks were ligated in various orders and numbers between the 5′-common sequence, T7, and the 3′-common sequence, Ex.

(4) Confirmation of the Mutant DNA Library

For the purpose of examining whether or not the PCR product obtained in (2) above had the sequence as predicted above, the DNAs band corresponding to T7-(X)⁶-Ex was excised and after cloning, the DNA sequencing was performed. First, the DNA band corresponding to T7-(X)⁶-Ex was purified using Wizard PCR Preps DNA Purification System (manufactured by Promega). Next, after addition of dA to the 3′-end using Taq DNA polymerase, DNA was cloned using TOPO TA Cloning KIT (Invitrogen). Cloning operations were the same as those described in (2) above, except that LB plates to which IPTG and X-gal had not been applied, were used.

After incubating the LB plate for 12 hours at 37° C., around 100 colonies were confirmed on the plate. Colony PCR was performed in respect of all colonies to examine whether insert DNA was contained or not. The colony PCR mixture (50 μl) comprises 20 pmol each of M13 forward and reverse primers that were provided with the Kit, 200 μM of dNTP, 1.5 mM MgCl₂, 1 unit of Taq DNA polymerase (manufactured by Toyobo Co., Ltd.), and 5 μl of the provided 10×Taq DNA polymerase buffer. As template. DNA, a colony suspended in sterilized water was used. Reaction conditions were as follows: Stage 1 (1 cycle), 95° C. for 5 minutes; Stage 2 (30 cycle), 95° C. for 30 seconds, 55° C. for 30 seconds, 72° C. for 30 seconds; Stage 3 (1cycle) 4° C. for 10 minutes. As a result of analysis of th colony PCR products by agarose electrophoresis, it was confirmed that 66 colonies comprised the insert DNA. These colony PCR products were purified using Wizard PCR Preps DNA Purification System (Manufactured by Promega), and nucleotide sequences were analyzed by a DNA sequencer (CEQ2000 DNA Analysis System, manufactured by Beckman Coulter, Inc.).

Results of analysis of the 66 sequences which were confirmed to be insert DNA are shown in Table 1. The numbers in the Table represent the block sequences given as DNA fragments 1 to 6. Of the 66 sequences, 64 sequences had a structure with T7 at the 5′-end, and Ex at the 3′-end, and building block DNAs ligated in various orders therebetween. That is, it was confirmed that a reaction had occurred just as predicted above. The content of the 66 sequences is as follows: 1 T7-(X)⁹-Ex sequence, 1 T7-(X)⁷-Ex sequence, 50 T7-(X)⁶-Ex sequences, 5 T7-(X)⁵-Ex sequences, 4 T7-(X)⁴-Ex sequences, 3 T7-(X)³-Ex sequences, and 2 sequences having the sequence T7-(X)⁷ but not having Ex on the 3′-side. Among these, there were only two pairs having identical sequences, and thus 64 different sequences were confirmed. That is, it was confirmed that many structural genes formed by ligation of 6 types of building block DNAs in various different orders were simultaneously synthesized in a single PCR. Because the sequences analyzed after cloning using E. coli, were only a small portion of the DNAs prepared by the PCR, it is predicted that many more types of structural gene are constructed in the mutant DNA library prepared by this PCR.

As a reason why DNA having shorter or longer lengths, other than T7-(X)⁶-Ex, were contained in the analyzed nucleotide sequence, it is considered that this was due to that a small number of DNA having differing lengths were present in the DNA band corresponding to T7-(X)⁶-Ex that was excised in the low melting point agarose electrophoresis. These DNA also had a sequence with T7 on the 5′-end, Ex on the 3′-end, and building block DNAs ligated in various orders therebetween.

Further, 2 types of sequence not having Ex at the 3′-end are predicted to have resulted from the ligated building block DNA that was supposed to be ligated inside, itself, acting as a primer. Further, with the sequence consisting of T7-3-4-6-5-1-2-5-Ex, a large deletion between 6-5 was observed. This was thought to be due to the fact that “gTACgACT” sequence from position 15 on the 5′-side of building block DNA 6, and the “gTACgACT” sequence from position 3 on the 5′-side of building block DNA S were identical sequences, and the recombination occurred between these two sequences.

Substitution was observed at 9 places in the 40299 base pairs that were analyzed. That is, the frequency of substitution was about 1 in 5000, which is a frequency that is about 2.5 to 5 times higher than the substitution frequency of a typical PCR using Vent DNA polymerase (Mattila et al. (1991) Nucl. Acids. Res., 19, 4967-4973). However, for the purpose of constructing a library for the evolutionary molecular engineering, this value was well within an acceptable range.

From the facts that, in the agarose electrophoresis of the PCR product in this Example, 15 or more DNA bands in ladder form were confirmed, and of the analyzed DNA sequence, most had different sequences, and mutations causing frame shift such as deletion, etc., did not occur, where 6 types of building block are used, 6¹⁵≈5×10¹¹ or more types of structural gene without stop codon can be constructed in a single PCR, as represented by the following formula:

$${\sum }_{n = 1}^{15} {6}^{n}$$

TABLE 1
Results of nucleotide sequence analysis of PCR products

| | | | | | |
|---|---|---|---|---|---|
| 112114 | 245 | 34514 | 432335 | 525155 | 645465^{a} |
| 113134 | 245256 | 343 | 446262 | 526655 | 646161 |
| 12156 | 254136 | 343611 | 45325 | 532 | 646362 |
| 12465 | 256541 | 345265 | 455124 | 5336155^{b} | 646551 |
| 134526 | 261136 | 346452 | 465222 | 542526 | 651656 |
| 136564 | 26212 | 3465125^{c} | 466325 | 563656 | 652636 |
| 145412 | 2641 | 35354 | 511533 | 565145 | 6552 |
| 146521 | 313542 | 354531 | 514231 | 614231 | 665115 |
| 2116 | 3212 | 363262 | 521314 | 623652 | 665145625 |
| 216431 | 324355 | 412351 | 521351 | 625362 | |
| 231531 | 332612 | 4263152^{b} | 523515 | 631234 | |

^{a}Sequences obtained from two colonies
^{b}Sequences without Ex at the 3′-end
^{c}Sequences with deletions between buildings block 6 and 5

### Example 2

(1) Preparation of Building block DNA

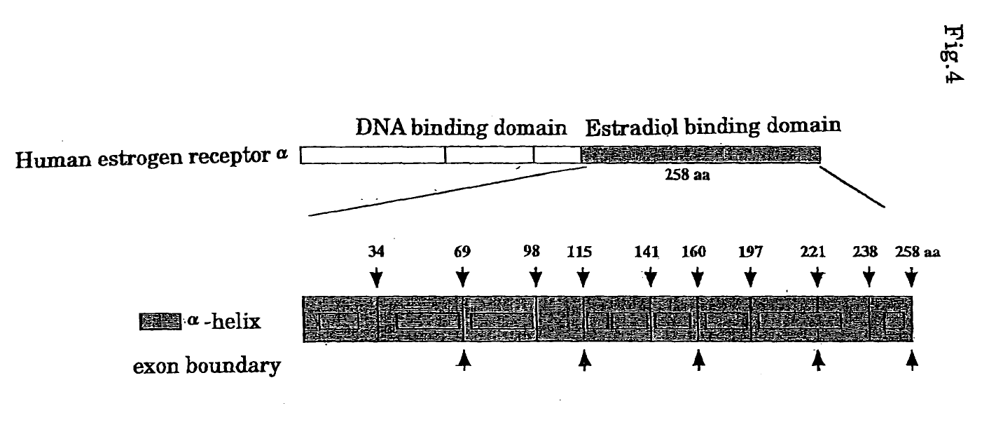

The ligand binding domain of human estrogen receptor α (hereinafter, also referred to as “hERαLBD”: Greene et al. (1986) Science, 231 (4742), 1150-1154; Brzozowski et al. (1997) Nature, 389 (6652), 753-758) is composed of 258 amino acid residues. The entire region of this hERαLBD was divided into 10 building blocks at the boundaries of the exons (Ponglikitmongkol et al.(1988) EMBO J., 11, 3385-3388) and the secondary structures on the conformational structure of the protein (Tanenbaum et al.(98) Proc. Natl. Acad. Sci. USA., 95(11), 5998-6003). Each building block DNA was prepared by amplification by PCR using a plasmid comprising hERαLBD as a template by using forward primers having homology to the 5′-side nucleotide sequence of the building block DNA fragments and reverse primers having homology to the 3′-side nucleotide sequence (prepared to order by Dateconcept), in such a way that each building block encoding, from the N-terminal side, 34, 35, 29, 17, 26, 37, 24, 17, 20 amino acid residues respectively (FIG. 4) (this plasmid was provided by Prof. G. L. Greene of Chicago University).

The prepared building blocks were designated as 1, 2, 3, 4, 5, 6, 7, 8, 9 and 10 from the N-terminal side. As primers, SEQ ID NOS: 25 and 26 were used for building block DNA 1, SEQ ID NOS: 27 and 28 for building block DNA 2, SEQ ID NOS: 29 and 30 for building block DNA 3, SEQ ID NO: 31 and 32 for building block DNA 4, SEQ ID NOS: 33 and 34 for building block DNA 5, SEQ ID NOS: 35 and 36 for building block DNA 6, SEQ ID NOS: 37 and 38, for building block DNA 7, SEQ ID NOS: 39 and 40 for building block DNA 8, SEQ ID NOS: 41 and 42 for building block DNA 9 and SEQ ID NOS: 43 and 44 were used for building block DNA 10. Further, apart from these building block DNAs, a DNA fragment comprising T7 promoter sequence as a 5′-end common sequence DNA fragment (T7OM: SEQ ID NO: 45), and a 3′-end common sequence DNA fragment (CBSHis: SEQ ID NO: 46) were prepared by chemical synthesis (entrusted to Dateconcept, Inc.). Common sequence DNA fragments were prepared by amplification PCR using these common sequence synthetic DNA fragments as a template, using a forward primer having homology to the 5′-side nucleotide sequence of each DNA fragment (T7OM: SEQ ID NO: 47; CBSHis: SEQ ID NO: 49) and a reverse primer having homology to the 3′-side nucleotide sequence (prepared to order by Dateconcept) (T7OM: SEQ ID NO: 48; CBSHis: SEQ ID NO: 50). The prepared DNA fragments having common sequences were designated T7OM for the 5′-side, and CBSHis for the 3′-side.

Primers used for preparation of building block DNA and common sequence DNA fragments were phosphorylated prior to PCR. The phosphorylation reaction solution (30 μl) comprises 20 μl of DNA solution (20 pmol/μl), 10 mM ATP, polynucleotide phosphate kinase (manufactured by New England Biolabs, Inc.), and 3 μl of the provided 10× reaction buffer. The phosphorylation reaction was performed at 37° C. for 12 hours,

PCR using phosphorylated primers was performed with a reaction solution (50 μl) comprising DNA solution (20 ng/μl) 1 μl, 20 pmol of each primer, 200 μM of dNTPs, 2.5 unit Pfu DNA polymerase (manufactured by STRATAGENE), and 5 μl of the provided 10×Pfu DNA polymerase reaction buffer, in the following reaction conditions: Stage 1 (1 cycle), 95° C. for 5 minutes; Stage 2 (30 cycles), 95° C. for 30 seconds, 56° C. for 30 seconds; and Stage 3 (1 cycle), 4° C. for 10 minutes. The above described 10 building block DNA fragments and 2 common sequence DNA fragments amplified by PCR were prepared using Wizard PCR Preps (manufactured by Promega), and dissolved in 60 μl of sterilized water.

(2) Preparation of Ligated Building Block DNA

Twelve types of DNA fragment prepared as described in (1) were ligated with following combinations using DNA ligase, and PCRs were performed using the ligated building block DNA fragments as templates, forward primers encoding sequences complementay to 5′-end of first building block DNA in ligated building block DNAs and reverse primers encoding sequences complementary to 3′-end of second building block DNA in the ligated building block DNAs: T7OM-1, T7OM-2, T7OM-3, T7OM-4, T7OM-5, T7OM-6, T7OM-7, T7OM-8, T7OM-9, T7OM-10, 1-2, 1-3, 1-4, 1-5, 1-6, 1-7, 1-8, 1-9, 1-10, 1-CBSHis, 2-3, 2-4, 2-5, 2-6, 2-7, 2-8, 2-9, 2-10, 2-CBSHis, 3-4, 3-5, 3-6, 3-7, 3-8, 3-9, 3-10, 3-CBSHis, 4-5, 4-6, 4-7, 4-8, 4-9, 4-10, 4-CBSHis, 5-6, 5-7, 5-8, 5-9, 5-10, 5-CBSHis, 6-7, 6-8, 6-9, 6,-10, 6-CBSHis, 7-8, 7-9, 7-10, 7-CBSHis, 8-9, 8-10, 8-CBSHis, 9-10, 9-CBS His

The solution for ligating building block DNA fragments by DNA ligase comprises 1 μl of DNA solution (10 ng/μl), 400 units of T4 DNA ligase (manufactured by New England Biolabs), 1 μl of the provided 10×T4 DNA ligase reaction buffer, and 6 μl of sterilized water, and the reaction was performed at 16° C. for 1 hour. The PCR reaction solution (50 μl) containing this ligated building block DNA fragment as a template, comprises 20 pmol of each primer, 200 μM of dNTPs, 1 μl of the above described DNA ligase reaction solution containing the ligated building block DNAs, 1.5 mM MgCl₂, 1 unit of Taq DNA polymerase (manufactured by Toyobo Co., Ltd..), and 5 μl of the provided 10×Taq DNA polymerase reaction buffer. PCR was performed in the following reaction conditions: Stage 1 (1 cycle), 95° C. for 5 minutes, Stage 2 (30cycles), 95° C. for 30 seconds, 60° C. for 30 seconds, and Stage 3 (1cycle), 4° C. for 10 minutes.

The obtained ligated building block DNA fragments described above were cloned using TOPO TA Cloning KIT (manufactured by InVitrogen). Cloning procedures were according to the protocol attached with the KIT (TOPO TA Cloning, Ver.K: Five minutes cloning of Taq polymerase-amplified PCR products, Invitrogen), and as a vector, one contained in the KIT was used. Specifically, first, 4 μl of PCR reaction solution of each ligated building block DNA fragment prepared as described above was mixed with 1 μl of the salt solution provided and 1 μl of PCR TOPO vector, and the mixture was incubated at 25° C. for 5 minutes.

Next, 2 μl of the above described reaction solution and 50 μl of the provided competent cells were mixed and incubated on ice for 30 minutes. Thereafter, after incubating for 30 seconds at 42° C., 250 μl of the provided SOC medium was added, and this was further incubated for 30 minutes at 37° C. 50 μl of this was applied to an LB plate having 50 μl/ml ampicillin added thereto, after application thereto of 24 μl of 40 μg/ml isopropyl-1-thio-β-galactoside galactose (IPTG), and 40 μl of 40 μg/ml X-gal solution.

After incubating the above described LB plate for 12 hours at 37° C., around 200 colonies were confirmed on the plate. Three blue colonies were selected for each ligated building block DNA, and it was confirmed by colony PCR that the target ligated building block was contained in the plasmid. Here, the reason why blue were selected was because all of the building block DNA had a nucleteotide sequence being a multiple of 3, and it was predicted that these would not cause a frame shift in lacZ gene and have no influence on the activity. The reaction solution for the above described colony PCR comprises 20 pmol of each of M13 forward and reverse primers, which were provided with TOPO TA Cloning KIT (manufactured by InVitrogen), 200 μM of dNTPs, 1.5 mM MgCl₂, 1 unit of Taq DNA polymerase (manufactured by Toyobo Co., Ltd.), and 5 μl of 10×Taq DNA polymerase reaction buffer that was provided, and a solution obtained by suspending the colony in sterilized water, and PCR was performed in the following reaction conditions: Stage 1 (1 cycle), 95° C. for 5 minutes; Stage 2 (30cycles), 95° C. for 30 seconds, 60° C. for 30 seconds, 72° C. for 30 seconds; Stage 3 (1 cycle), 4° C. for 10 minutes.

After analyzing this colony PCR reaction solution by agarose gel electrophoresis, and confirming that the target ligated building block DNA fragments had been amplified, the product was purified using Wizard PCR Preps DNA Purification System (manufactured by Promega), and the nucleotide sequence was analyzed using a DNA sequencer (CEQ2000 DNA Analysis System: manufactured by Beckman Coulter, Inc.). The DNA sequencing confirmed that there were no mutations such as substitutions, insertions, or deletions, etc.

Using a plasmid comprising ligated building block DNA confirmed here to have the correct nucleotide sequence as a template, ligated building block DNAs were prepared again by colony PCR using a forward primer having homology to the 5′-end sequence of the first building block DNA of each ligated building block DNA indicated in (1), and a reverse primer having homology to the 3′-end sequence of the second building block DNA of each ligated building block DNA indicated in (1). The PCR reaction solution (50 μl) comprises 20 pmol of each primer, 200 μM of dNTPs, 3.75 unit Pfu DNA polymerase (manufactured by STRATAGENE), and 5 μl of the provided 10×Pfu DNA polymerase reaction buffer. PCR was performed in the following reaction conditions: Stage 1 (1cycle), 95° C. for 5 minutes; Stage 2 (30 cycles), 95° C. for 30 seconds, 56° C. for 30 seconds; and Stage 3 (1cycle), 4° C. for 10 minutes.

Ligated building block DNA fragments obtained from the PCR reaction solution in which the above described ligated building block DNA was prepared, were concentrated by ethanol precipitation and dissolved in 20 μl sterilized water. Thereafter, the DNA fragments were separated by low melting point agarose gel electrophoresis, and target DNA bands were excised. DNA was extracted from this agarose gel fragment using phenol/chloroform. This was ethanol precipitated and dissolved in 60 μl of sterilized water. The concentration of each DNA solution containing ligated building block DNA obtained was determined by measuring adsorption at 260 nm. The ligated building block DNAs obtained here were used as template DNA for the next PCR for constructing a mutant DNA library.

(3) Classification of Ligated Building Block DNAs

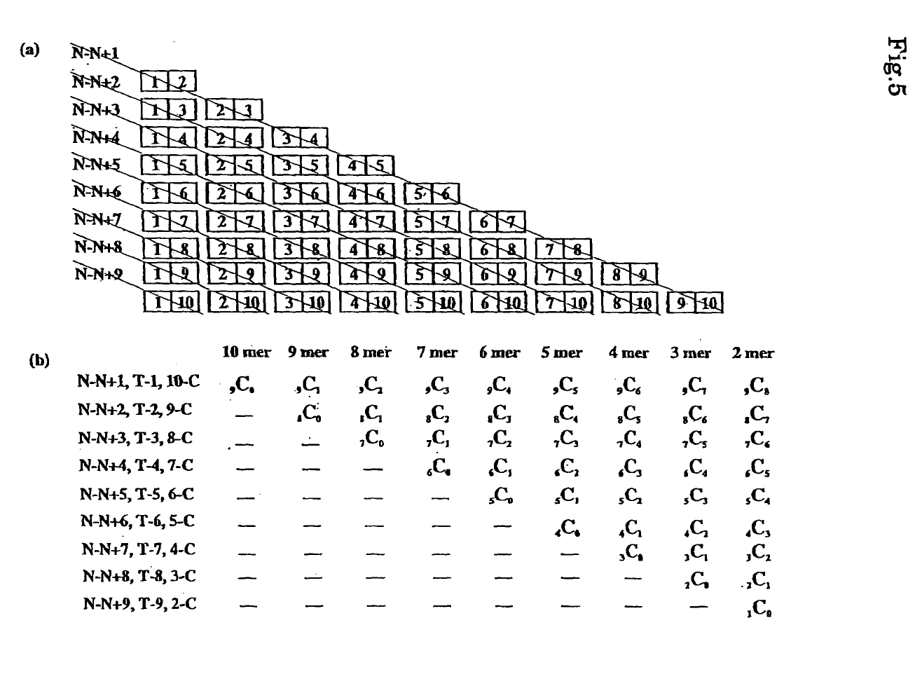

All of the ligated building block DNAs obtained in (2) above, were classified according to the building block encoded by them. Classification was performed based on the number of intervening building blocks between the first and second building block DNA in a ligated building block DNA prepared as described above, in the structural gene DNA in which they are found. Here, where X is the number of intervening building blocks between the first building block DNA and the second building block DNA in the structural gene DNA in which they are present, the prepared ligated building block DNA could be classified into 9 types, from X=0 to X=8 (FIG. 5(a)). (4) Construction of a mutant DNA library where 8 building block DNAs are ligated

A mutant DNA library wherein 8 building block DNAs were ligated was prepared using the ligated building block DNA group classified as described in (3) above as a template. In respect of each ligated building block DNA group, the proportion of each ligated building block DNA group to the total amount of DNA to be used in PCR was determined by applying the following formula (1) to the total number (L) of building blocks into which the structural gene was segmented, i.e. 10, and the difference (M) between the total number of building block DNAs and the number of building block DNAs included in the DNA to be prepared, i.e. 2, and the number of intervening building block DNAs (X) between the first building block DNA and the second building block DNA in the structural gene DNA in which they are present:

_{L-X-1}C_{M-X} (1)

The result of the calculation indicated following ration; 36 for the ligated building block DNA group classfied into X=0 according to the classification described in (3) above; 36 for T7OM-1;36 for 10-CBSHisX; 8 for the X=1 ligated building block DNA group; 8 for T7OM-2; 8 for 9-CBSHisX; 1 for the X=2 ligated building block DNA group; 1 for T7OM-2; and 1 for 8-CBSHisX.

As a specific amount of DNA, because a total amount of 675 fmol of DNA was used in PCR to prepare the mutant DNA library, the total amount of DNA of the X=0 ligated building block DNA group becomes 180 fmol, i.e. 26 fmol each for ligated building block DNAs (1-2, 2-3, 3-4, 5-6, 7-8, 8-9, 9-10) falling in this classification, 180 fmol for T7OM-1, and 180 fmol for 10-CBSHisX. The total amount of DNA for the X=1 ligated building block DNA group becomes 40 fmol, i.e., 5 fmol each for ligated building block DNAs in this classification (1-3, 2-4, 3-5, 4-6, 5-7, 6-8, 7-9, 8-10), 40 fmol for T7OM-2 and 40 fmol for 9-CBSHisX. The total amount of DNA for the X=2 ligated building block DNA group becomes 5 fmol, i.e. 0.7 fmol for each ligated building block DNA in this classification (1-4, 2-5, 3-6, 4-7, 5-8, 6-9, 7-10), 5 fmol for T7OM-3, and 5 fmol for 8-CBSHisX. DNA was mixed in the above proportions and used as PCR template DNA.

The PCR reaction solution (50 μl) comprises, as primers, 20 pmol each of T7OM forward primer and CBPHis reverse primer, 675 fmol of the above described mixed template DNA, 200 μM dNTPs, 1.25 units of KOD Dash DNA polymerase (manufactured by Toyobo Co., Ltd..), and 5μl of the provided 10×KOD Dash DNA polymerase reaction buffer, and PCR was performed in the following reaction conditions: Stage 1 (1 cycle), 95° C. for 5 minutes; Stage 2 (20 cycles), 95° C. for 30 seconds, 60° C. for 30 seconds, 72° C. for 3 minutes; and Stage 3 (1 cycle), 4° C. for 10 minutes.

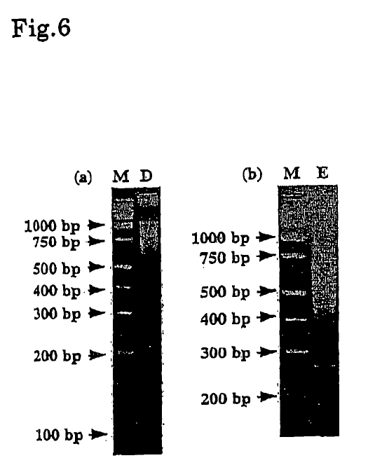

After concentrating the above described PCR product by ethanol precipitation, it was analyzed by low melting point agarose gel electrophoresis. As a result, strong DNA band was confirmed in the region corresponding to the intended molecular weight (FIG. 6(a)).

(5) Construction of a Mutant DNA Library Where 5 Building Block DNAs are Ligated

A mutant DNA library was prepared wherein 5 building block DNAs were ligated, by using the ligated building block DNA group classified in (3) above as a template. In respect of each ligated building block DNA group, proportions of each ligated building block DNA group to the total amount of DNA to be used in PCR were determined by applying the above described formula (1); the total number (L) of ligated building blocks into which the structural gene was segmented, i.e. 10, and the difference (M) between the total number of building blocks and the number of building block DNA included in the DNA to be prepared, i.e. 5, and the number of intervening building blocks (X) between the first building block DNA and the second building block DNA in the structural gene DNA in which they are present.

The result of calculation indicated following ration; 126 for the ligated building block DNA group falling into classification X=0 according to the classification described in (3) above; 126 for T7OM-1; 126 for 10-CBSHisX; 70 for the X=1 ligated building block DNA group; 70 for T7OM-2; 70 for 9-CBSHisX; 35 for the X=2 ligated building block DNA group; 35 for T7OM-2; 35 for 8-CBSHisX; 15 for the X=3 ligated building block DNA group; 15 for T7OM-4; 15 for 7-CBSHisX; 5 for the X=4 ligated building block DNA group; 5 for T7OM-5; 5 for 6-CBSHisX; 1 for the X=5 ligated building block DNA group; 1 for T7OM-6; and 1 for 5-CBSHisX.

As a specific amount of DNA, because a total amount of 1260 fmol DNA was used in the PCR in order to prepare the mutant DNA library, the total amount of DNA for the X=0 ligated building block DNA group becomes 210 fmol, i.e. 30 fmol of each ligated building block DNA in this classification (1-2, 2-3, 3-4, 5-6, 7-8, 8-9, 9-10), 210 fmol of T7OM-1 and 210 fmol of 10-CBSHisX. The total amount of DNA for the X=1 ligated building block DNA group becomes 117 fmol, i.e. 14.6 fmol of each ligated building block DNA in this classification (1-3, 2-4, 3-5, 4-6, 5-7, 6-8, 7-9, 8-10), 117 fmol of T7OM-2 and 117 fmol of 9-CBSHisX. The total amount of DNA for the X=2 ligated building block DNA group becomes 58 fmol, i.e. 8 fmol of each ligated building block DNA in this classification (1-4, 2-5, 3-6, 4-7, 5-8, 6-9, 7-10), 58 fmol of T7OM-3, and 58 fmol of 8-CBSHisX. The total amount of DNA for the X=3 ligated building block DNA group becomes 25 fmol, i.e. 4 fmol for each ligated building block DNA in this classification (1-5, 2-6, 3-7, 4-8, 5-9, 6-10), 25 fmol for T7OM-4 and 25 fmol for 7-CBSHisX. The total amount of DNA for the X=4 ligated building block DNA group becomes 8.3 fmol, i.e. 1.6 fmol for each ligated building block DNA in this classification (1-6, 2-7, 3-8, 4-9, 5-10), 8.3 fmol for T7OM-5, and 8.3 fmol for 6-CBSHisX. The total amount of DNA for the X=5 ligated building block DNA group becomes 1.7 fmol, i.e. 0.4 fmol for each ligated building block DNA in this classification (1-7, 2-8, 3-9, 4-10), 1.7 fmol for T7OM-6 and 1.7 fmol for 5-CBSHisX. DNA was mixed in the above proportions and used as PCR template DNA.

The PCR reaction solution (50 μl) comprises as primers, 20 pmol each of T7OM forward primer and CBPHis reverse primer, 1260 fmol of the above described mixed template DNA, 200 μl dNTPs, 1.25 units of KOD Dash DNA polymerase (manufactured by Toyobo Co., Ltd..) and 5 μl of the provided 10×KOD Dash DNA polymerase reaction buffer. PCR was performed in the following reaction conditions: Stage 1 (1 cycle), 95° C. for 5 minutes; Stage 2 (20 cycles), 95° C. for 30 seconds, 60° C. for 30 seconds, 72° C. for 3 minutes; and Stage 3 (1 cycle), 4° C. for 10 minutes.

After concentrating the above described PCR products by ethanol precipitation, they were analyzed by low melting point agarose gel electrophoresis. As a result, strong DNA band was confirmed in the region corresponding to the intended molecular weight (FIG. 6(b)).

(6) Confirmation of the Mutant DNA Libraries

In order to confirm whether or not the PCR products obtained in (4) and (5) above possess the intended sequence, regions exhibiting strong DNA bands obtained by the above described low melting point agarose gel electrophoresis were excised, and the DNA was extracted and purified using Wizard PCR Preps DNA Purification System (manufactured by Promega). Next, after adding dA to the 3′-end using Taq DNA polymerase, the DNA was cloned using TOPO TA Cloning KIT (manufactured by Invitrogen) in the same manner as in (2) above. At this time, LB plates to which IPTO and X-Gal had not been applied, were used.

After incubating this LB plate for 12 hours at 37° C., 100 or more colonies could be confirmed on the plate. In respect of more than 40 colonies, the nucleotide sequences of the PCR productsDNA prepared in (4) and (5) contained in the plasmids were analyzed by the same method as in (2) above.

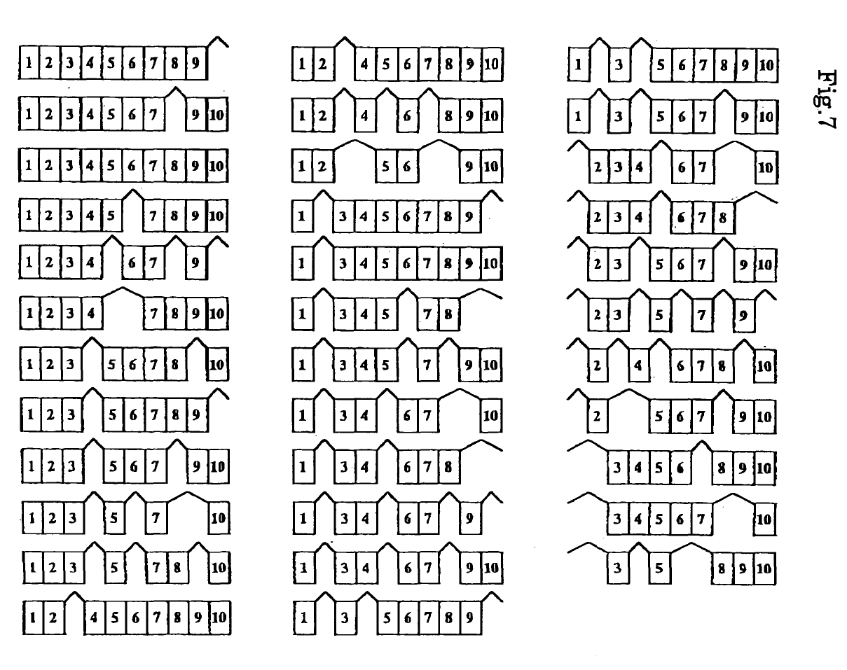

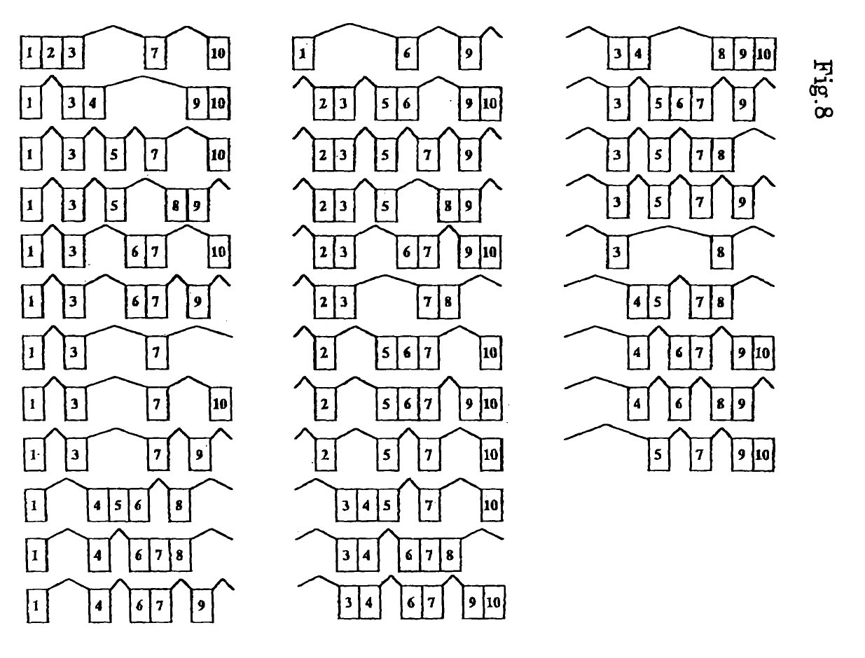

Regarding the PCR product described in (4) above (where 8 building block DNAs are ligated), nucleotide sequences were analyzed in respect of 38 clones. The results are shown in FIG. 7. Further, regarding the PCR product described in (5) above (where 5 building blocks are ligated), nucleotide sequences of 33 clones were analyzed. The results are shown in FIG. 8. The DNA fragments obtained in either PCR, all possessed T7OM sequence at the 5′-end and CBSHis sequence at the 3′ end, and the internal building block DNAs possessed a structure such that they were ligated variously while preserving their order in the structural gene from upstream to downstream. The number of each building block DNA appearing in the nucleotide sequences of the 71 DNA fragments analyzed were as follows: 1/39, 2130, 3/52, 4/34, 5/40, 6/43, 7/56, 8/32, 9/43, 10/40. This confirmed that no clear bias in frequency of the building block DNAs used was observed.

Further, among the DNA fragments analyzed as described above, there was only one set of sequences indicated in FIG. 7 which had the same sequence, In respect of substitution, in the 71 clones, there were only 60 instances of substituted nucleotides among 47533 nucleotides. This rate of mutation (1.3×10⁻³) was judged to be sufficiently low and no obstacle for the construction of the library. In fact, among 50 substitutions, only two changed the codon to stop codons. Further, there were 2 types of sequences formed by ligation of unintended regions. One occurred by induction of homologous recombination at “atgatc” which was in both above described building block DNAs 1 and 2. The other occurred by induction of homologous recombination between “ccaggga” and “ccagtga” which were in the above described building block DNA S and building block DNA 4, respectively. In sequences other than these no mutations which would cause a frame shift, i.e. deletions and insertions, were observed. That is, it was indicated that most portion of the DNA contained in the PCR-generated mutant DNA library of the present invention, preserved the correct reading frame.

Among the sequences shown in FIG. 7, there were 3 clones of DNA fragments in which 5 building block DNAs were ligated, 10 clones of DNA fragments in which 6 building block DNAs were ligated, 9 clones of DNA fragments in which 7 building block DNAs were ligated, 10 clones of DNA fragments in which 8 building block DNAs were ligated, and 2 clones of DNA fragments in which 9 building block DNAs were ligated, and 1 clone of a DNA fragment in which 10 building block DNAs were ligated. Further, among the sequences indicated in FIG. 8, there was one clone of DNA fragments in which 2 building block DNAs were ligated, 2 clones of DNA fragments in which 3 building block DNAs were ligated, 9 clones of DNA fragments in which 4 building block DNAs were ligated, 17 clones of DNA fragments in which 5 building block DNAs were ligated, and 4 clones of DNA fragments in which 6 building block DNAs were ligated.

The above result indicates that while DNA fragments consisting of the number of building block DNAs that is more or less than the intended number of building block DNAs, DNA fragments consisting of the target number of building block DNAs are of the greatest number, and therefore, it is possible to construct a mutant DNA library sufficient for achieving an object of the present invention which is the creation of a useful protein by application of evolutionary molecular engineering to the library.

### Example 3

A mutant DNA library was prepared by Error-prone PCR.

When ligated building blocks were mixed and PCR was performed by the same operations as in (1) to (3) and (5) in Example 2, manganese chloride was added to the PCR reaction solution at a concentration of 0 to 1 mM. The nucleoride sequence of the amplified DNAs were analyzed in the same manner as described in (6) of Example 2. The results are shown in Table 2. It was clear that the frequency of nucleotide substitution increased almost linearly relative to the increase of manganese ion concentration. Therefore, it was clarified that when seeking to construct a library with a given substitution rate, manganese ions should be added in accordance with the data in Table 2.

TABLE 2

| | | | substitution/ |
|---|---|---|---|
| MnCl₂ | analyzed base pairs | number of substitutions | 1000 bp |
| 0 | 47533 | 60 | 1.3 |
| 3 | 3669 | 5 | 1.4 |
| 5 | 3129 | 6 | 1.9 |
| 7 | 2424 | 5 | 2.1 |
| 150 | 2751 | 12 | 4.4 |
| 250 | 3344 | 22 | 6.6 |
| 350 | 3933 | 23 | 5.8 |
| 425 | 3663 | 38 | 10.4 |
| 475 | 3810 | 45 | 11.5 |
| 500 | 3234 | 59 | 18.2 |
| 750 | 2916 | 84 | 28.8 |
| 1000 | 2202 | 75 | 34 |

Industrial Applicability

According to the method of the present invention, there is provided a method of constructing a group of genes formed by ligation of a plurality of DNA fragments encoding any amino acid sequence in various orders and lengths. By selecting a desired protein from a mutant protein library prepared from the mutant DNA library, and further using evolutionary molecular engineering, this method is extremely useful for creating a protein having a new function by combining natural protein parts (building blocks) such as seconndary structures, modules and motifs.

Further, proteins having downscaled molecular weights can be obtained from the mutant protein library prepared from the alternative splicing library of the present invention. Denaturation of relatively small globular proteins having a molecular weight of 20,000 or less, is known to be reversible, and even if once denatured, can have their functions restored by return to suitable conditions. Further, proteins having such a property, have strong resistance to heat and organic solvents, as is represented by ribonuclease. Further, the yield of purified products in bioprocesses using such enzymes is high. Because mutant proteins obtained from the alternative splicing library of the present invention can be downsized from any protein while preserving its function, their applicability as proteins will broaden.

1. A method of constructing a DNA library comprising:
(a) ligating building block DNAs in any combination;
(b) selecting and mixing the obtained ligated building block DNAs such that all ligated building block DNAs to be used have an end sequence that overlaps with an end sequence of at least one other type of ligated building block DNA; and
(c) performing Polymerase Chain Reaction using the mixture of ligated building block DNAs as a template to obtain a DNA library comprising 2 or more clones.

2. The method according to claim 1 wherein, in step (b), the ligated building block DNAs are selected and mixed such that all ligated building block DNAs to be used have end sequences that overlap with the end sequence of at least one other type of ligated building block DNA, and such that either the 5′-end or 3′-end sequence of at least one type of ligated building block DNA overlaps with an end of at least two other types of ligated building block DNA.

3. The method according to claim 1 or 2 wherein the mixture of ligated building block DNAs is a mixture of ligated building blocks formed by ligation of 2 building block DNAs in all combinations of the building block DNAs used.

4. The method according to claim 1, wherein the building block DNAs are obtained from a structural gene encoding a protein which was segmented into L units (where L is an integer of 3 or more); wherein the method of ligation is such that the 3′ end of any building block DNA is ligated to the 5′-end of a building block DNA existing further toward the C-terminal side of the protein than the above building block DNA, in such a way that the amino acid sequence of each building block is preserved; and wherein the mixture of ligated building block DNAs is prepared by the classification according to X (where X is an integer between 0 and L-2, inclusive), which is the number of intervening building block DNAs between these block DNAs ligated in the structural gene, and by mixing with the proportions fulfilling the following formula (1):
_{L-X-1}C_{M-X} (1)
wherein, L and X have the same meaning as that described above, and M represents the difference between the total number of building blocks and the number of building blocks possessed by the mutant DNA to be prepared.

5. The method according to claim 1, wherein the building block DNA results from segmentation of each DNA encoding a group of proteins having a similar 3-dimensional structure, but differing amino acid sequences, into L building blocks (where, L is an integer of 3 or more); wherein the method of ligation is such that the 3′-end of any building block DNA is ligated to the 5′-end of a building block DNA existing further toward the C-terminal side of the group of proteins than the above building block DNA in such a way that the amino acid sequence of each building block is preserved, and wherein the mixture of ligated building block DNAs is prepared by the classification according to X (where X is an integer between 0 and L-2, inclusive), which is the number of intervening building block DNAs between these block DNAs ligated in the structural gene, and by mixing with the proportions fulfilling the following formula (1):
_{L-X-1}C_{M-X} (1)
wherein the formula, L and X have the same meaning as that described above, and M represents the difference between the total number of building blocks and the number of building blocks possessed by the mutant DNA to be prepared.

6. The method according to any one of claims 1 to 5 wherein the Polymerase Chain Reaction is conducted in the presence of primers having sequences complementary to the building block DNA intended to form both ends of the DNA to be constructed.

7. The method according to any one of claims 1 to 6 wherein the Polymerase Chain Reaction is performed in the presence of ligated building block DNA comprising a common DNA sequence, and a primer having a sequence complementary to the common DNA sequence.

8. A mutant DNA library obtained by the method of any one of claims 1 to 7.

9. A protein library obtained by incorporating the DNA library according to claim 8 into an expression vector, transforming a host cell with the expression vector, culturing the transformant, and collecting proteins from the culture product.

10. An RNA library obtained by transcription of the DNA library according to claim 8.

11. A method of preparing a protein library, which comprises expressing the DNA library according to claim 8 by use of the cell free transcription and/or translation system or the RNA library according to claim 10 by a cell free transcription system.

12. A protein library obtained by the method of claim 11.

13. The library of nucleic acid-protein ligated molecule whose proteins are encoded by DNA or RNA from the DNA library according to claim 7 or from RNA library according to claim 9.

14. A method of obtaining a protein which comprises selecting a protein having a desired property using the library according to claim 9, 12 or 13.

15. A protein obtained by the method according to claim 14.

16. A method of obtaining a protein, which comprises:
(a) preparing DNA encoding a protein according to claim 15;
(b) introducing mutations into the DNA;
(c) amplifying the DNA into which mutations were introduced
(d) preparing a protein library by transcription/translation of the amplified DNA; and
(e) obtaining a protein having a desired property using the library.

17. A protein obtained by the method according to claim 16.

18. A method of obtaining DNA encoding the protein according to claim 15 or 17.

19. DNA obtained by the method according to claim 18.
